(19) United States
(12) Patent Application Publication
(10) Pub. No.: US 2025/0273298 A1
(43) Pub. Date: Aug. 28, 2025

(54) METHODS AND SYSTEMS FOR GENOMIC BASED PREDICTION OF VIRUS MUTATION

(71) Applicant: THE UNIVERSITY OF CHICAGO, Chicago, IL (US)

(72) Inventor: Ishanu CHATTOPADHYAY, Chicago, IL (US)

(21) Appl. No.: 18/036,528

(22) PCT Filed: Nov. 17, 2021
(86) PCT No.: PCT/US2021/059616

§ 371 Date: May 11, 2023

Related U.S. Application Data
(60) Provisional application No. 63/198,849, filed on Nov. 17, 2020.

(51) Int. Cl. G16B 30/10

(52) U.S. Cl. CPC G16B 30/10

(57) ABSTRACT

A method includes receiving a first plurality of aligned genomic sequences of a virus from a database. The aligned genomic sequences have a first common background. The method includes calculating a Qnet for each genomic sequence of the first plurality of aligned genomic sequences. The Qnet for each sequence is calculated by calculating a conditional inference tree for each index of the aligned genomic sequences using other indices in the aligned genomic sequences as predictive features, and calculating predictors for indices that were used as predictive features when calculating the conditional inference tree for each index.

### CROSS-REFERENCE TO RELATED APPLICATIONS

This application is a U.S. National Stage Entry of International Patent Application No. PCT/US2021/059616, filed Nov. 17, 2021, which claims the benefit of priority to U.S. Provisional Patent Application Ser. No. 63/198,849, filed Nov. 17, 2020, the entire contents of which is hereby incorporated by reference in their entirety.

### STATEMENT REGARDING FEDERALLY SPONSORED RESEARCH & DEVELOPMENT

This invention was made with government support under HR0011-18-9-0043 awarded by the Department of Defense (DOD) DARPA. The government has certain rights in the invention.

### REFERENCE TO AN ELECTRONIC SEQUENCE LISTING

The contents of the electronic sequence listing (Sequence_Listing_37997-123.txt; Size: 9,733 bytes; and Date of Creation: May 3, 2023) is herein incorporated by reference in its entirety.

### TECHNICAL FIELD

This disclosure relates generally to the computational prediction of jump-likelihood between viruses, and, more specifically, to systems and methods for predicting dominant circulating strains and inter-species jump risk of viruses.

### BACKGROUND

With estimated mortality rates significantly higher compared to that of the seasonal flu, the COVID-19 (SARS-CoV-2) pandemic of 2020 may be one of the most destructive pandemics of the past century. Improved preparation for the next pandemic is desirable. The ability to predict emergence of novel pathogens, such as SARS-CoV-2, with an actionable timeline may help to reduce the negative impact of future pandemics. Current surveillance paradigms, while capable of mapping disease ecosystems, are limited in their ability to address such a challenge. Habitat encroachment, climate change, and other ecological factors increase the odds of novel viruses “jumping” from a host species to humans, resulting in pandemics such as that associated with SARS-CoV-2. At least some known efforts aimed at tracking and modeling these effects to date have not been able to successfully quantify future risk of emergence of a specific strain from a specific host species. Existence of viral diversity in hosts such as bats, swines or wild ducks, while important, might not transparently map to emergence risk, and may not address the problem at hand.

One of the key hurdles in addressing this problem has been the ability to quantitatively assess the risk of emergence from strains that circulate in the wild. Current techniques generally do not have the tools necessary to numerically compute the likelihood of a biological sequence replicating in the wild and spontaneously giving rise to another by random chance. Currently the similarity between two genomic sequences is typically measured by how many mutations it takes to change one sequence to the other, e.g. the number of mutations that make an avian flu strain human-adapted. However, without taking into account the odds of those mutations occurring in the wild, such a measure may not accurately measure the true jump-risk between species. The odds of one sequence mutating to another is a function of not just how many mutations they are apart to begin with, but also how specific mutations incrementally affect fitness. Without taking into account the constraints arising from the need to conserve function, assessing the jump-likelihood is subjective and inaccurate.

Current surveillance protocols are also generally insufficient for predicting dominant viral strains in seasonal epidemics, such as Influenza A (flu), that will circulate in the human population during any given year. Currently, the World Health Organization (WHO) decides on flu vaccine recommendations primarily from bio-surveillance data collected in the previous flu season and afterward. However, there is no established method to accurately model how the virus is expected to evolved, and it has generally been assumed that such dynamics are either too complex to model or are entirely random.

The development of computational methods and systems that are able to calculate the precise likelihood of one viral sequence mutating into another would allow for better risk assessment of inter-species jump of novel viruses and better prediction of dominant strains of seasonal viruses.

This background section is intended to introduce the reader to various aspects of art that may be related to the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### BRIEF DESCRIPTION

One aspect of this disclosure is a method that includes receiving a first plurality of aligned genomic sequences of a virus from a database. The aligned genomic sequences have a first common background. The method includes calculating a Qnet for each genomic sequence of the first plurality of aligned genomic sequences. The Qnet for each sequence is calculated by calculating a conditional inference tree for each index of the aligned genomic sequences using other indices in the aligned genomic sequences as predictive features, and calculating predictors for indices that were used as predictive features when calculating the conditional inference tree for each index.

Another aspect of this disclosure is a method that operates on a plurality of aligned genomic sequences of an organism from a database, to calculate a “Qnet” for that organism. The Qnet is a representation of computationally inferred dependencies between non-colocated mutations recorded in the database for the organism selected, as present in the wild around the time of collection of the sequences, subject to the selection pressures in effect at that time. The Qnet is calculated by estimating a conditional inference tree for each index of the aligned genomic sequences using other indices in the aligned genomic sequences as predictive features. The Qnet is thus a collection of predictors, where the target variable for a component prediction of the Qnet shows ups as predictive features for other predictors. Thus, the Qnet is a recursively dependent forest of predictors.

Various refinements exist of the notion of Qnet in relation to the above-mentioned aspects. Further features may also be incorporated in the above-mentioned aspects as well. These refinements and additional features may exist individually or in any combination. For instance, various features discussed below in relation to any of the illustrated examples may be incorporated into any of the above-described aspects, alone or in any combination.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a schematic showing how sequence variations are used to derive a new biological metric, q-distance, for comparing differences between mutating sequences.

FIG. 1B is a schematic showing how q-distance can be used to calculate the likelihood of a jump between strains and to model future emergence risk.

FIG. 2A is a schematic showing a portion of the recursive forest underlying the Qnet for human Influenza A hemagglutinin (HA) during the 2018-2019 season.

FIG. 2B is a Qnet dependency graph for SARS-CoV-2 spike protein from the 2019 COVID-19 pandemic.

FIG. 2C is a Qnet dependency graph for Influenza A HA from the 2009 Swine Flu pandemic.

FIG. 3A is a bar graph comparing the accuracy of the WHO- and Qnet-predicted dominant strain sequence for Influenza A H1N1 HA in the southern hemisphere.

FIG. 3B is a bar graph comparing the accuracy of the WHO- and Qnet-predicted dominant strain sequence for Influenza A H1N1 neuraminidase (NA) in the southern hemisphere.

FIG. 3C is a bar graph comparing the accuracy of the WHO- and Qnet-predicted dominant strain sequence for Influenza A H3N2 HA in the southern hemisphere.

FIG. 3D is a bar graph comparing the accuracy of the WHO- and Qnet-predicted dominant strain sequence for Influenza A H3N2 NA in the southern hemisphere.

FIG. 3E is a bar graph comparing the accuracy of the WHO- and Qnet-predicted dominant strain sequence, using a multi-cluster approach, for Influenza A H1N1 NA in the southern hemisphere.

FIG. 3F is a bar graph comparing the accuracy of the WHO- and Qnet-predicted dominant strain sequence, using a multi-cluster approach, for Influenza A H3N2 NA in the southern hemisphere.

FIG. 3G is a bar graph comparing the accuracy of the WHO- and Qnet-predicted dominant strain sequence for Influenza A H1N1 HA in the northern hemisphere.

FIG. 3H is a bar graph comparing the accuracy of the WHO- and Qnet-predicted dominant strain sequence for Influenza A H1N1 NA in the northern hemisphere.

FIG. 3I is a bar graph comparing the accuracy of the WHO- and Qnet-predicted dominant strain sequence for Influenza A H3N2 HA in the northern hemisphere.

FIG. 3J is a bar graph comparing the accuracy of the WHO- and Qnet-predicted dominant strain sequence for Influenza A H3N2 NA in the northern hemisphere.

FIG. 3K is a bar graph comparing the accuracy of the WHO- and Qnet-predicted dominant strain sequence, using a multi-cluster approach, for Influenza A H1N1 NA in the northern hemisphere.

FIG. 3L is a bar graph comparing the accuracy of the WHO- and Qnet-predicted dominant strain sequence, using a multi-cluster approach, for Influenza A H3N2 NA in the southern hemisphere.

FIG. 4A is a sequence comparison of the WHO-predicted, Qnet-predicted, and actual dominant H1N1 HA strain for the northern hemisphere in 2018-2019.

FIG. 4B is a sequence comparison for the WHO-predicted, Qnet-predicted, and actual dominant H1N1 HA strain for the northern hemisphere in 2019-2020.

FIG. 4C is a sequence comparison for the WHO-predicted, Qnet-predicted, and actual dominant H1N1 HA strain for the northern hemisphere in 2018-2019.

FIG. 4D is a sequence comparison for the WHO-predicted, Qnet-predicted, and actual dominant H1N1 HA strain for the northern hemisphere in 2016-2017.

FIG. 4E is a sequence comparison for the WHO-predicted, Qnet-predicted, and actual dominant H1N1 HA strain for the southern hemisphere in 2014-2015.

FIG. 4F is a sequence comparison for the WHO-predicted, Qnet-predicted, and actual dominant H3N2 HA strain for the northern hemisphere in 2015-2016.

FIG. 4G is a molecular structure of the HA protein highlighting residues that deviate between WHO- and Qnet-predicted sequences.

FIG. 5A is a pair of bar graphs comparing the log-likelihood of spontaneous jump from (i) different animal hosts to the SARS-CoV-2 sequences of the 2019 pandemic and (ii) from specific species to their nearest SARS-CoV-2 neighbors.

FIG. 5B is a map showing the habitats of the top four most frequently occurring species from FIG. 5A (ii).

FIG. 5C is a graph plotting the log-likelihood of a jump between various sequences and their nearest neighbors against the year of collection.

FIG. 5D is a map showing the density of habitat overlap from FIG. 5B.

FIG. 6 is a phylogenetic tree derived from q-distance of various coronaviruses.

FIG. 7 is a plot showing the distribution around the seasonal dominant strain for various influenza strains.

FIG. 8A is a phylogenetic tree for various coronaviruses based on q-distance.

FIG. 8B is a phylogenetic tree for various coronaviruses based on standard edit distance.

FIG. 9A is a distribution space map comparing the distribution of Influenza HA q-sampled mutations compared to random mutations.

FIG. 9B is a graph plotting the p-blast scores for q-sampled and random mutants against known sequences.

FIG. 9C is a graph plotting the distance between mutant quasi-species vs. iteration step for q-distance and edit distance metric.

FIG. 9D is a graph plotting variance of pairwise distances for q-sampled and random mutants compared to known sequences.

FIG. 9E is a bar graph plotting the probability per year of BLAST match for q-sampled and random mutants.

FIG. 10A is a scatter plot showing the membership degree of initial hCOV-19 strains.

FIG. 10B is a distribution plot of the membership degrees for hCoV-19 strains collected on Mar. 31, 2020 (black) and Apr. 3, 2020 (red).

FIG. 10C is a bar graph showing the average membership p-values for hCoV-19 strains collected on different dates.

FIG. 11 is a schematic showing an exemplary method of evaluating jump-likelihood probability between different viral strains.

FIG. 12 is a schematic of a system for carrying out the method of FIG. 11.

FIG. 13 is a schematic of a computing device for the system of FIG. 12.

Although specific features of various embodiments may be shown in some drawings and not in others, this is for convenience only. Any feature of any drawing may be referenced and/or claimed in combination with any feature of any other drawing.

Unless otherwise indicated, the drawings provided herein are meant to illustrate features of embodiments of the disclosure. These features are believed to be applicable in a wide variety of systems comprising one or more embodiments of the disclosure. As such, the drawings are not meant to include all conventional features known by those of ordinary skill in the art to be required for the practice of the embodiments disclosed herein.

### DETAILED DESCRIPTION

The following detailed description illustrates embodiments of the disclosure by way of example and not by way of limitation. Embodiments of the systems and methods described herein may predict the circulating strain of evolving pathogens, such as Influenza A (flu), with actionable lead time to inform vaccine design. The disclosed embodiments may be expected to predict dominant strains of future seasonal epidemics with more accuracy than the World Health Organization (WHO) recommendations used currently in flu shot compositions. Embodiments of the systems and methods described herein may also calculate the risk of evolving pathogens to jump between species, enable the origin of past pandemics to be traced and future pandemics to be predicted. While the exemplary embodiments include prediction of the dominant seasonal strain of Influenza A and tracing the species origin of SARS-CoV-2, the described embodiments are in no way meant to be limiting. Embodiments of the systems and methods described herein may be applied to any viral sequences, granted the sequences are similar enough to conduct a sequence alignment and there is sufficient diversity of observed strains.

Embodiments of the systems and methods described herein provide for the calculation of the likelihood that a viral sequence will mutate into another, leading to emergence of new viral strains either within species (e.g., novel dominant seasonal strains of influenza) or between species (e.g., novel human SARS-CoV-2). Embodiments of the systems and methods described herein provide for building a computational model, or Qnet, for providing these predictions. A suite of customized machine learning algorithms may be used to infer the Qnet from aligned genomic sequences sampled from similar populations, for example, hemagglutinin (HA) from human Influenza A in year 2008, or the spike protein from all bat betacoronaviruses. The Qnet can predict the nucleotide distribution over the base alphabet (the four nucleic acid bases ATGC) at any specific index, conditioned on the nucleotides making up the rest of the sequence of the gene or genome fragment under consideration.

As described herein, the Qnet can learn to predict the mutational variations at each index of the genomic sequence using other indices as features, ultimately uncovering a recursive dependency structure. Collectively, these inter-dependent predictors represent the constraints that shape evolutionary trajectories driven by selection.

Embodiments of the systems and methods described herein provide for a Qnet-derived metric for calculating similarity between species, referred to as the q-distance. The q-distance can be defined as the square-root of the Jensen-Shannon (JS) divergence of the conditional distributions from one sequence to another, wherein the conditional distributions are produced by the Qnet, and averaged over the entire sequence. As a function of the q-distance, the bounds on the explicit probability of a spontaneous jump between nearby variants can be computed.

Embodiments of the systems and methods described herein demonstrably improve strain predictions for Influenza A vaccines compared to historic WHO strain predictions that form the basis for current vaccine recommendations. The recommendations produced by the systems and methods described herein are repeatedly closer to the true dominant strain, illustrating the ability of these systems and methods to correctly predict evolutionary trajectories. High season-to-season genomic variation in the key Influenza capsidic proteins is driven by two opposing influences: the need to conserve function limiting random mutations, and hyper-variability to escape recognition by neutralizing antibodies. Even a single residue change in the surface proteins might dramatically alter recognition characteristics, brought about by unpredictable changes in local or regional properties such as charge, hydropathy, side chain solvent accessibility.

Embodiments of the systems and methods described herein provide for a Qnet predicted strain (QNT) that is more likely to be closer to the dominant strain of Influenza A than the WHO predicted strain over the past two decades, and almost consistently over the last decade. The Qnet predicted strain is able to predict residues present in the dominant strain that are not predicted by the WHO. These residues are largely localized within the receptor binding domain (RBD), with >57% occurring within the RBD on average. When the WHO-predicted strain deviates from the Qnet predicted/dominant strain matched residue, the “correct” residue is often replaced in the WHO recommendation with one that has a very different side chain, hydropathy and/or chemical properties, suggesting deviations in recognition characteristics. Because circulating strains are almost always within a few edits of the dominant strain, hosts vaccinated with the Qnet recommendation may be more likely to have season-specific antibodies that are more likely to recognize a larger cross-section of the circulating strains.

Embodiments of the systems and methods described herein demonstrate that the deviations in the Qnet predicted and WHO predicted strain residues are largely localized in the HA1 subunit of the HA molecular structure with the most frequent deviations occurring around the ≈200 loop, the ≈220 loop, the ≈180 helix, and the ≈100 helix, in addition to some residues in the HA2 subunit (≈49 and ≈124). The residues most impacted in the HA1 subunit (the globular top of the fusion protein) have been repeatedly implicated in receptor binding interactions. Embodiments of the systems and methods described herein are able to fine tune the future influenza vaccine recommendation over the state of the art, largely by modifying residue recommendations around the RBD and structures affecting recognition dynamics.

Embodiments of the systems and methods described herein can calculate the likelihood of viral strains collected across disparate host species to give rise to the observed SARS-CoV-2 strains, and offer new insights into the SARS-CoV-2 origin of the 2020 pandemic backed by precise numerical assessments. In the context of the origin problem of the 2020 pandemic, the state of the field regarding SARS-CoV-2 ancestry is still developing, with emerging consensus on horseshoe bats of Chinese origin as the potential host of the progenitor sequence. This narrative is primarily driven by observed edit-distance and motif similarities to bat coronavirus (RaTG13, accession MN996532.1) detected in R. affinis from the Yunan province. However, this consensus does not explain the existence of a polybasic furin cleavage site on the spike protein which is absent in RaTG13 and related betacoronaviruses, but do occur in other human coronaviruses including HKU130.

Embodiments of the systems and methods described herein provide for a q-distance analysis that demonstrates not only the progenitor host potential of R. affinis, but also that a related species R. sinicus is a slightly more probable source for SARS-CoV-2. Also, it can be demonstrated that several other closely related horseshoe bats including R. ferrumequinum and R. monoceros, and other bats such as T. pachypus, V. superans, and P. abramus are also potential progenitor hosts. In addition, rodents such as R. argentiventer, N. confucianus, and A. agrarius have credible potential as hosting a SARS-CoV-2 ancestor.

In some embodiments, the collection times of animal samples may be plotted against the average lower log-likelihood bound on spontaneous jump to SARS-CoV-2 sequences to demonstrate dependence of the jump probability on collection date. This dependence suggests risk-progression over time. Embodiments of the systems and methods described herein demonstrate that the early risky sequences of SARS-CoV-2 are exclusively from rodents, and the risk elevates through late 2018, with the majority of the hosts switching from rodents to bats to human coronaviruses (OC43 and HKU1). This progression can be further highlighted by a LOWESS regression (local polynomial fit to the data points), which shows an almost constant gradient of risk elevation over the past decade. Additionally, habitats of the top species that pose this risk can be overlapped, suggesting a normalized habitat distribution consistent with the presumed ground zero of the outbreak (Wuhan, China).

The quantitative assessments provided by the systems and methods described herein are not enabled by the prior art, and suggest that the evolution of SARS-CoV-2 began in rodents and jumped to bats, with final maturation in humans. The gradual elevation of risk through multiple host species, the overlapping habitats of those species, and the ability to quantify the minimum bounds on jump probability enabled by the present disclosure provide significant utility in preparing for future pandemics.

Embodiments of the systems and methods described herein provide for a data-driven metric, q-distance, to track subtle deviations in sequences, and quantify jump risk of risky viral pathogens. The systems and methods described herein demonstrate ability to predict future strains of Influenza via subtle variations in a limited set of immunologically important residues, suggesting that the systems and methods provided herein may be useful in preempting and actionably mitigating the next pandemic.

### Example Method for Building Viral Prediction Model (Qnet) and Calculating Q-Distance

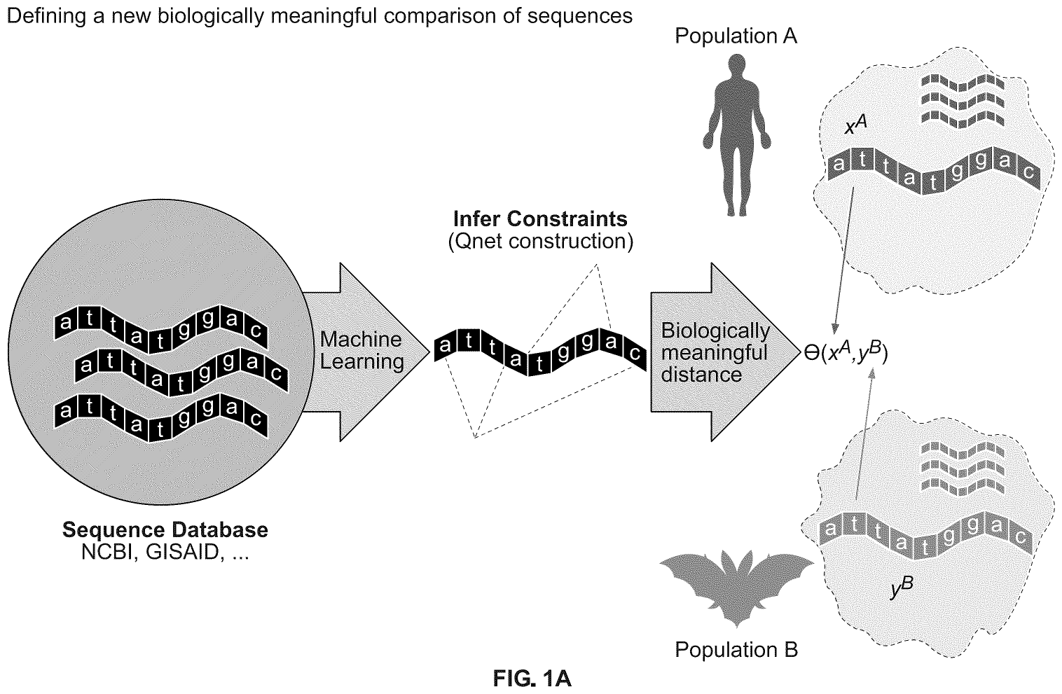

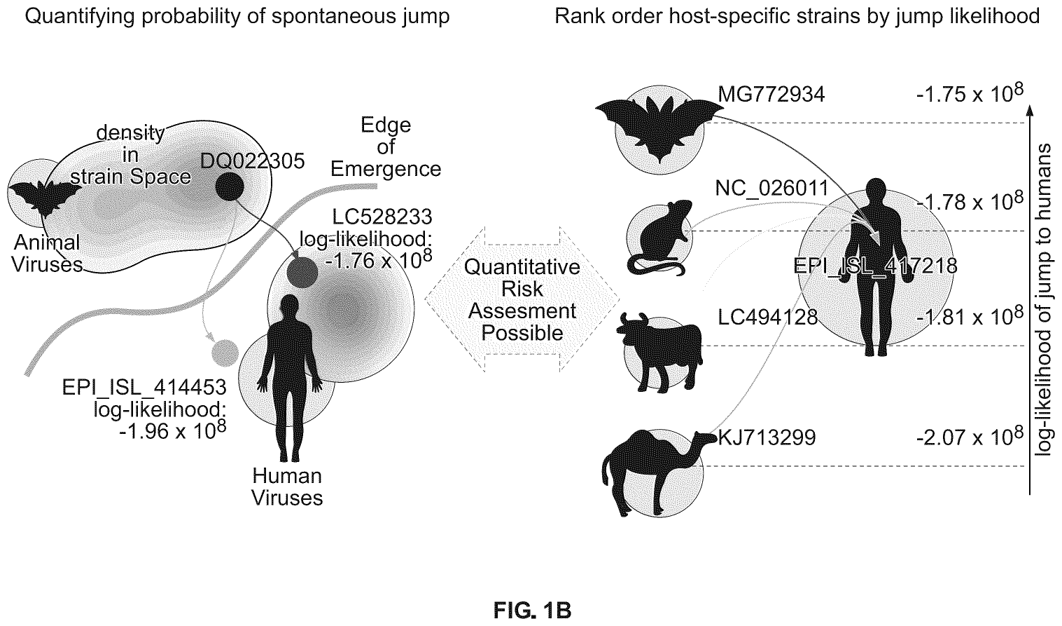

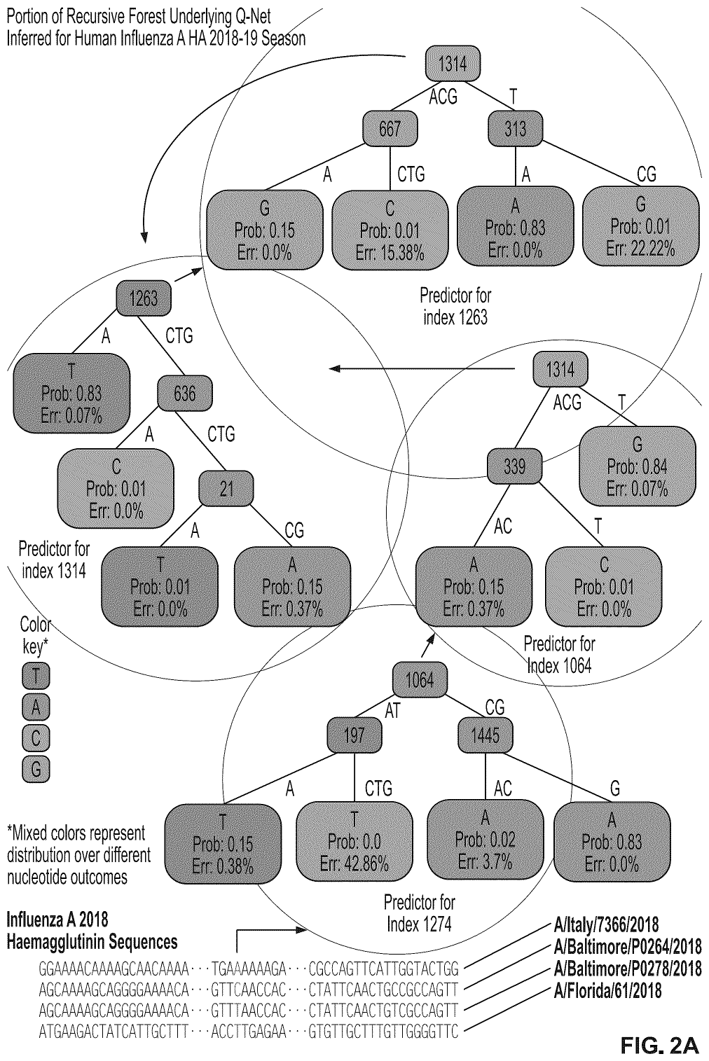

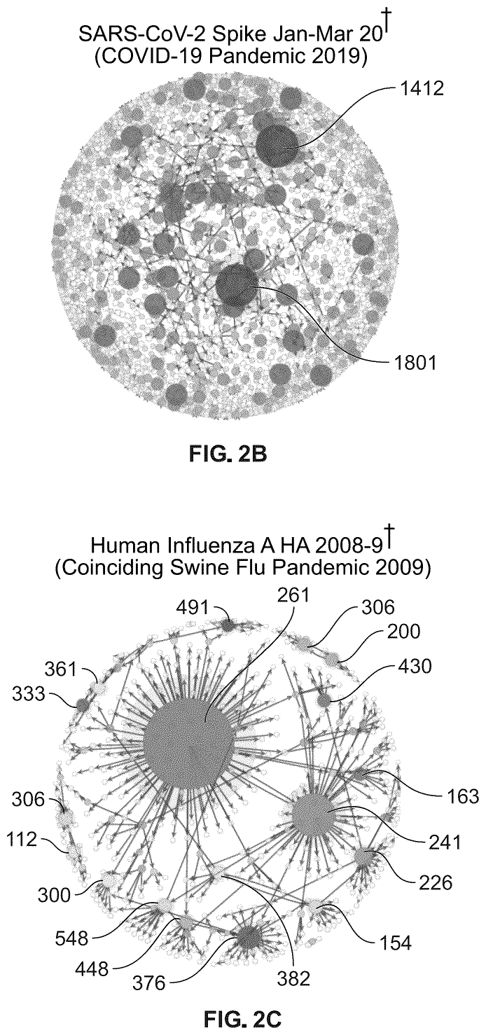

In an example embodiment, relevant coding sequences can be collected pertaining to key genes implicated in cellular entry from two public databases (NCBI and GISAID, see e.g., TABLE 4 below for number of distinct sequences used). For example, an excess of 30,000 distinct sequences for betacoronaviruses and Influenza A can be used, focusing on three genes or proteins. For each organism, a network of dependencies between individual mutations can be revealed through subtle variations of the aligned sequences. These dependencies can then be used to define an organism-specific model referred to as the quasi-species network, or Qnet (see e.g., FIG. 1A-FIGS. 1B and 2A-FIG. 2C).

FIG. 1A-FIG. 1B is a series of schematics that provide an overview of the Qnet algorithm capability to quantify risk and rank-order strains. FIG. 1A shows that sequence variations observed in large databases can be used to distill evolutionary constraints on a genomic sequence to induce a biology-aware metric for comparing subtle differences in mutating sequences. This metric (q-distance) adjusts to specific organisms, background populations and selection pressures, and reflects the true likelihood of a spontaneous jump from one sequence to the other. This sequence level metric can be used to compute distances between a sequence and a population, and two populations. FIG. 1B illustrates that bounds on the exact likelihood of a spontaneous jump between strains can be calculated and rank-order strains observed in a diverse set of hosts to accurately model future emergence risk.

FIG. 2A-FIG. 2C illustrates the Qnet computation scheme. FIG. 2A shows, beginning with aligned sequences, a conditional inference tree can be calculated for index 1274, which involves indices 1064, 1445, 197 as predictive features. These features are automatically selected by the algorithm, as being maximally predictive of the base at 1274. Then, predictors for each of these predictive indices are calculated, e: g: the inference tree computed for index 1064 is shown, which involves index 1314 and 339 as features. Continuing, the predictor for 1314 involves indices 1263, 636 and 21, and that for 1263 involves 1314, 667 and 313. Note that recursive dependencies arise automatically: the predictor for 1263 depends on 1314, and that for 1314 depends on 1263. FIG. 2B and FIG. 2C are Qnet dependency graphs for SARS-CoV-2 spike protein and Influenza A HA respectively, illustrating the distinct patterns of mutational constraints inferred. Both HA in Influenza A and the spike protein in SARS-CoV-2 are implicated in viral entry into host cells, and crucial for host specificity of infections. Additionally, the inferred structures underscore the significantly more complex dependencies in SARS-CoV-2 compared to Influenza A.

In some embodiments, only sequences of high fitness may be observed, and only a small subset of viable sequences may ever be isolated. For example, a single 10 KB observed sequence represents a single observation in a 10,000 dimensional space: thus, enough data points may not be collected to exhaustively model the set of epistatic dependencies for any realistic genome length. Nevertheless, the systems and methods described herein demonstrate that sufficient sequences have been accumulated to yield meaningful results, at least for some RNA viruses with high mutational rates that reveal enough of the hidden constraints. The ability of the systems and methods described herein to quantitatively contrast sequence similarity addresses key aspects the viral surveillance and prediction problem, allowing for precise comparisons to be made there were not possible before.

In some embodiments, a suite of customized machine learning algorithms can be employed to infer the Qnet from aligned genomic sequences sampled from similar populations, e.g., HA from Human Influenza A in year 2008, or the spike protein from all bat beta coronaviruses. For the machine learning algorithms, conditional inference trees may be used to predict each index as a function of the other indices, which were chosen automatically by the inference algorithm while optimizing the best split in the course of the decision tree construction. For example, sequences for the spike(S) protein on betacoronaviruses, which plays a crucial role in host cellular entry, and the Hemagglutinin (HA) and Neuraminidase (NA) for Influenza A (for subtypes H1N1 and H3N2), which are key enablers of cellular entry and exit mechanisms respectively, can be used. The sequences may be obtained from sequence databases, for example, National Center for Biotechnology Information (NCBI) virus and GISAID databases. In some embodiments, a total of 30,204 sequences can be used (see e.g., TABLE 4).

The Qnet of the present disclosure can predict the nucleotide distribution over the base alphabet (the four nucleic acid bases ATGC) at any specific index, conditioned on the nucleotides making up the rest of the sequence of the gene or genome fragment under consideration. A q-distance can then be defined as the square-root of the Jensen-Shannon (JS) divergence of the conditional distributions from one sequence to another, averaged over the entire sequence. In defining the q-distance, the mutational variations at the individual indices of a genomic sequence are not assumed to be independent (see e.g., FIG. 1A). Irrespective of whether mutations are truly random, since only certain combinations of individual mutations are viable, individual mutations across a genomic sequence replicating in the wild appear constrained, which is what can be explicitly modeled in the systems and methods described herein. The mathematical form of the q-distance metric is not arbitrary: JS divergence is a symmetricised version of the more common Kullbeck Leibler (KL) divergence between distributions, and among different possibilities, the q-distance is the simplest metric such that the likelihood of a spontaneous jump is provably bounded above and below by simple exponential functions of the q-distance.

As an example, consider a set of random variables X={Xᵢ} with i=∈{1, . . . N}, each taking value from the respective sets Σᵢ. A sample xϵΠ₁^{N}Σᵢ is an ordered N-tuple, consisting of a realization of each of the variables Xᵢ with the i^{th} entry xᵢ being the realization of random variable Xᵢ. The notation x₋ᵢ and x^{i,σ} is used to denote:

$${x}_{- i} {=}^{△} {x}_{1} , … , {x}_{i - 1} , {x}_{i + 1} , … , {x}_{N} \\ {x}^{i , σ} {=}^{△} {x}_{1} , … , {x}_{i - 1} , σ , {x}_{i + 1} , … , {x}_{N} , σ ∈ {\sum }_{i}$$

D(S) can be used to denote the set of probability measures on a set S, e.g., D(Σᵢ) is the set of distributions on Σᵢ. Note that X defines a random field over the index set {1, . . . N}. Also, to highlight the biological relevance, the sample x is herein referenced as an amino acid or nucleotide sequence, wherein the entry at each index is identified with the corresponding protein residue or the nucleotide base pair.

For a random field X={Xᵢ}, indexed by i=∈{1, . . . N}, the Qnet can be defined to be the set of predictors:

$${Φ}_{i} : {\prod }_{j \neq i} {\sum }_{j} \to ( {\sum }_{i} ) ,$$

where for a sequence x, Φᵢ(x₋ᵢ) estimates the distribution of Xᵢ on the set Σᵢ. Conditional inference trees can then be used as models for predictors, although more general models may also be used.

Next, the Qnet model can be used to calculate q-distance, a novel, ‘biology-aware’ metric that calculates the distance between sequences incorporating information about biological context and evolutionary constraints. The q-distance, informed by the dependencies modeled by the inferred Qnets, can adapt to the specific organism, allelic frequencies, and nucleotide variations in the background population. Because the role of epistatic effects in phenotypic change is well-recognized, these effects can be incorporated in a numerically precise manner to compute bounds on the likelihood of specific strains giving rise to target variants.

The q-distance can be defined as the square-root of the Jensen-Shannon (JS) divergence of the conditional distributions from one sequence to another, obtained from the Qnet model, averaged over the entire sequence. Given two sequences x, y∈Π₁^{N}Σᵢ, such that x, y are drawn from the populations P, Q inducing the QnetΦ^{P}, Φ^{Q}, respectively, a pseudo-metric θ(x, y) can be defined as follows:

$$θ ( x , y ) {=}^{△} {E}_{i} ( {𝕁}^{\frac{1}{2}} ( {Φ}_{i}^{P} ( {x}_{- i} ) , {Φ}_{i}^{Q} ( {y}_{- i} ) ) ) ,$$

where [symbol] (.,.) is the Jensen-Shannon divergence and Eᵢ indicates expectation over the indices. The square-root in the definition of the q-distance arises naturally from the provable bounds, and is dictated by the form of Pinsker's inequality, ensuring that the distances along a path in a constructed phylogeny sum linearly. Therefore in some embodiments, standard algorithms can used for phylogeny construction.

Importantly, the q-distance defined above is technically a pseudo-metric because distinct sequences can induce the same distributions over each index, and thus evaluate to have a zero distance. This is a desirable feature, because the distance should not be sensitive to changes that are not biologically relevant: not all sequence variations brought about by substitutions are equally important or likely. Even with no selection pressure, random variations at an index can occur if such variations do not affect the replicative fitness. Under that scenario, the corresponding Φᵢ will predict a flat distribution no matter what the input sequence is, thus contributing nothing to the overall distance. Furthermore, even if two strains x, y have the same entry at some index i, the remaining residues might induce different distributions Φᵢ based on the remote dependencies, e.g., the entries in x₋ᵢy₋ᵢ.

In some embodiments, the q-distance between two sequences may change if the background populations change, and not the sequences themselves (see e.g., TABLE 2 for examples, where the distance between two specific Influenza A H1N1 Hemagglutinin sequences vary when they are assumed to be collected in different years), and sequences might have a large q-distance and a small edit distance, and vice versa (although on average the two distances tend to be positively correlated, see e.g., TABLE 3). Therefore, in some embodiments, a new Qnet may be constructed whenever the background populations are expected to be substantially different. For example, separate Qnets may be constructed for betacoronavirus S protein sequences isolated from bats, rodents, cattle, non-SARS-CoV-2 human betacoronaviruses, and SARS-CoV-2 strains. As another example, for tracking drift in Influenza A, a seasonal Qnet may be constructed for each subtype and protein that is considered. Because the q-distance assumes aligned sequences of identical length (although gaps arising from alignment are acceptable and are modeled as missing data), the Qnet framework is applicable to closely related sequences, and is well-suited to track subtle changes in evolving viral populations.

In some embodiments, it may be considered whether sequences come from two different background populations, Q, e.g., if the induced Qnets Φ^{P}Φ^{Q} are different. For example, if Qnets are constructed for H1N1 Influenza A separately for the collection years 2008 and 2009, then the same exact sequence collected in the respective years might have a non-zero distance between them, reflecting the fact that the background population the sequences arose from are different, inducing possibly different expected mutational tendencies.

Embodiments of the systems and methods described herein provide for measuring the q-distance between a sequence and a population and between two populations. The q-distance between populations can be defined using the notion of Hausdorff metric between sets:

$$∀ x ∈ P , y ∈ Q , \\ θ ( x , Q ) = {min}_{y ∈ Q} θ ( x , y ) \\ θ ( P , Q ) = max \{ {max}_{x ∈ P} θ ( x , Q ) , {max}_{y ∈ Q} θ ( y , P ) \}$$

Embodiments of the systems and methods described herein also provide for a quantitative test of how well the Qnet represents the data, whether predictors need to be recalculated, and whether there are sufficiently many sequences. In one implementation, an explicit membership test can formulated to quantitatively test these parameters. Given a population P inducing the Qnet Φ^{P} and a sequence x, the membership probability of x can be calculated as:

$${ω}_{x}^{P} {=}^{△} Pr ( x ∈ P ) = {\prod }_{j = 1}^{N} ( {Φ}_{j}^{P} ( {x}_{- j} ) {\vert}_{{x}_{j}} )$$

Note that xⱼ is the j^{th} entry in x, and is thus an element in the set Σⱼ. Since the most pertinent case is when Σⱼ is a finite set, Φⱼ^{P}(x₋ⱼ)|xⱼ is the entry in the probability mass function corresponding to the element of Σⱼ which appears at the j^{th} index in sequence x. This calculation can be carried out for a sequence x known to be in the population P as well, which allows for a membership degree ωₓ^{P} to be defined. If X is a random field representing a population P, e.g., X=x is a randomly drawn sequence from P, then the membership degree ω^{P} is a function of the random variable X:

$${ω}^{P} ( X ) {=}^{△} {\prod }_{j = 1}^{N} ( {Φ}_{j}^{P} ( {X}_{- j} ) {\vert}_{{X}_{j}} )$$

ω^{P} takes values in the unit interval [0,1], and the probability x is a member of the population P is ω^{P}(X=x), denoted briefly as ωₓ^{P} or ω_{w} if P is clear from context. Since ω^{P}(X) is a random variable, sets of sequences can then be computed that better represent the population P, and ones that are on the fringe. In some embodiments, evaluation can be performed using a pre-specified significance-level if a particular sequence is not from the population P, thus identifying if predictors Φ need to be recomputed, or if the base population needs to be split. In some embodiments, a hypothesis testing scenario can be set up to determine if sequences are indeed from a test population, as follows.

For example, given a population P, inducing a Qnet Φ^{P}, and a sequence x, the null hypothesis can be assumed to be x∉P. The null hypothesis can be rejected with a pre-specified significance α, if

$$Pr ( {ω}^{P} ( X ) ≧ {ω}^{P} ( X = x ) ) ≦ α$$

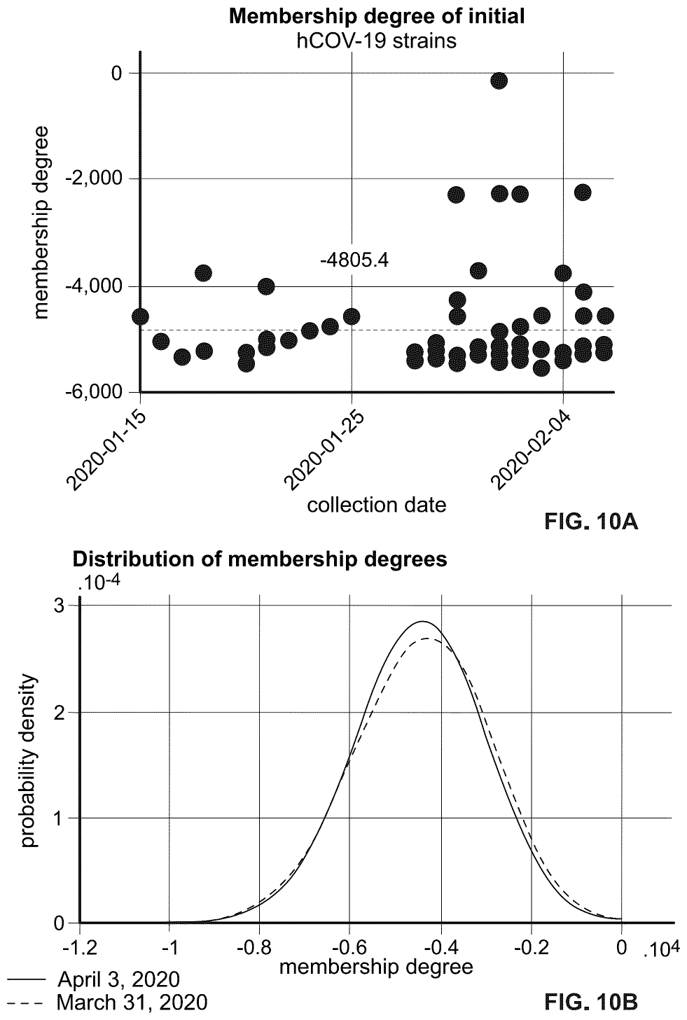

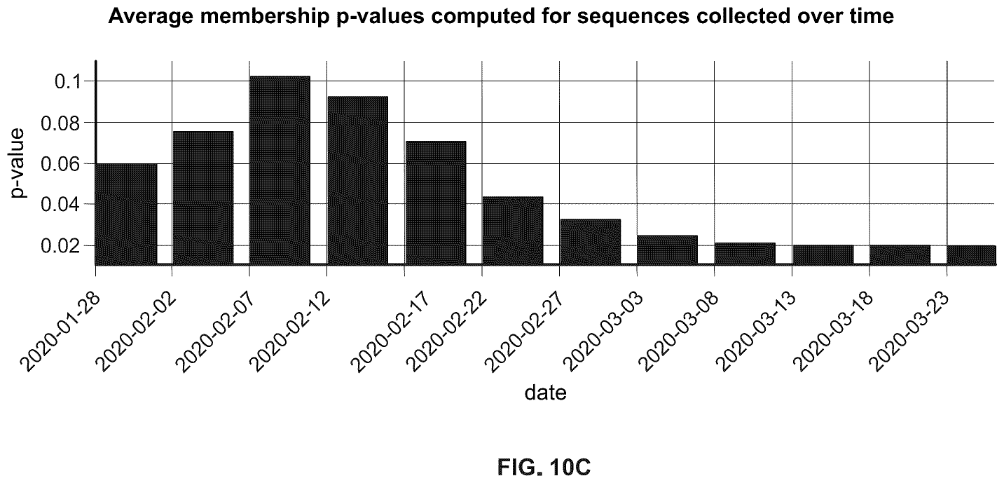

In some embodiments, the fraction of newly observed sequences that do not reject the null hypothesis can then be used as an estimate of the species-specific divergence in population characteristics. In an example embodiment, the membership degrees are calculated for the SARS-CoV-2 sequences in the early days of the pandemic, with respect to the constructed Qnet, and illustrated in FIG. 10A-FIG. 10C. The membership degree quantifies the likelihood that a test sequence actually is generated by the inferred model (e.g., the Qnet). In this example, the distribution of membership degrees is demonstrated to be very stable, and exhibits almost no change when more sequences are added (see e.g., FIG. 10B). In addition, as more sequences are collected, the p-value improves (see e.g, FIG. 10C), and stabilizes to about 0.02, demonstrating the validity of the model.

In some embodiments, the mathematical intuition behind relating the q distance to jump-probability is illustrated by the prediction of a biased outcome when a fair coin is tossed sequentially. With an overwhelming probability, such an experiment with a fair coin should result in roughly equal number of heads and tails. However, “large deviations” can happen, and the probability of such rare events is quantifiable with existing theory. Embodiments of the systems and methods described herein demonstrate that the likelihood of a spontaneous transition of a genomic sequence to a substantially different variant by random chance may also be similarly bounded, provided the Qnet as an estimated model of the evolutionary constraints.

In some embodiments, the Qnet framework described herein provides for rigorous computation of the bounds on several quantities of interest. The fundamental bound is on the probability of a spontaneous change of one strain to another, brought about by chance mutations. While any sequence of mutations is equally likely, the “fitness” of the resultant strain, or the probability that it will even result in a viable strain, is not. Thus the necessity of preserving function dictates that not all random changes are viable, and the probability of observing some trajectories through the sequence space are far greater than others. In some embodiments, the Qnet framework allows these constrained dynamics to be explored, as revealed by a sufficiently large set of genomic sequences.

With the exponentially exploding number of possibilities in the sequence space, it is computationally intractable to exhaustively model these dynamics. Nevertheless, possibilities can be constrained using the patterns distilled by the Qnet construction. As an example, given a sequence x of length N that transitions to a strain y∈Q, the following bounds exist at significance level α:

$${ω}_{y}^{Q} {e}^{\frac{\sqrt{8} {N}^{2}}{1 - α} θ ( x , y )} ≧ Pr ( x \to y ) ≧ {ω}_{y}^{Q} {e}^{- \frac{\sqrt{8} {N}^{2}}{1 - α} θ ( x , y )} ,$$

where ω_{y}^{Q} is the membership probability of strain y in the target population Q, and θ(x, y) is the q-distance between x, y. Using Sanov's theorem on large deviations, the probability of spontaneous jump from strain x∈P to strain y∈Q, with the possibility P≠Q, is given by:

$$Pr ( x \to y ) = {{\prod }_{i = 1}}^{N} ( {Φ}_{i}^{P} ( {x}_{- i} ) {\vert}_{{y}_{i}} )$$

Writing the factors on the right hand side as:

$${Φ}_{i}^{P} ( {x}_{- i} ) {\vert}_{{y}_{i}} = {Φ}_{i}^{{Q}_{}} ( {y}_{- i} ) {\vert}_{{y}_{i}} ( \frac{{Φ}_{i}^{P} ( {x}_{- i} ) {\vert}_{{y}_{i}}}{{Φ}_{i}^{Q} ( {y}_{- i} ) {\vert}_{{y}_{i}}} )$$

It can be noted that Φᵢ^{P}(x₋ᵢ), Φᵢ^{Q}(y₋ᵢ) are distributions in the same index i, hence:

$$\vert {{Φ}_{i}^{P} ( {x}_{- i} )}_{{y}_{i}} - {{Φ}_{i}^{Q} ( {y}_{- i} )}_{{y}_{i}} \vert ≦ {\sum }_{{y}_{i} ∈ {\sum }_{i}} \vert {{Φ}_{i}^{P} ( {x}_{- i} )}_{{y}_{i}} - {{Φ}_{i}^{Q} ( {y}_{- i} )}_{{y}_{i}} \vert$$

Using a standard refinement of Pinsker's inequality, and the relationship of Jensen-Shannon divergence with total variation, the following results:

$${θ}_{i} ≧ \frac{1}{8} {\vert {{Φ}_{i}^{P} ( {x}_{- i} )}_{{y}_{i}} - {{Φ}_{i}^{Q} ( {y}_{- i} )}_{{y}_{i}} \vert}^{2} \vert 1 - \frac{{{Φ}_{i}^{Q} ( {y}_{- i} )}_{{y}_{i}}}{{{Φ}_{i}^{P} ( {x}_{- i} )}_{{y}_{i}}} \vert ≦ \frac{1}{{a}_{0}} \sqrt{8 {θ}_{i}} ,$$

where a₀ is the smallest non-zero probability value of generating the entry at any index. This parameter is related to statistical significance of the bounds. First, the lower bound can be formulated as follows:

$$log ( {{\prod }_{i = 1}}^{N} \frac{{Φ}_{i}^{P} ( {x}_{- i} ) {\vert}_{{y}_{i}}}{{Φ}_{i}^{Q} ( {y}_{- i} ) {\vert}_{{y}_{i}}} ) = {\sum }_{i} log ( \frac{{Φ}_{i}^{P} ( {x}_{- i} ) {\vert}_{{y}_{i}}}{{Φ}_{i}^{Q} ( {y}_{- i} ) {\vert}_{{y}_{i}}} ) ≧ {\sum }_{i} ( 1 - \frac{{{Φ}_{i}^{Q} ( {y}_{- i} )}_{{y}_{i}}}{{{Φ}_{i}^{P} ( {x}_{- i} )}_{{y}_{i}}} ) ≧ \frac{\sqrt{8}}{{a}_{0}} {\sum }_{i} {θ}_{i}^{1 / 2} = - \frac{\sqrt{8} N}{{a}_{0}} θ$$

Similarly, the upper bound may be derived as:

$$log ( {{\prod }_{i = 1}}^{N} \frac{{Φ}_{i}^{P} ( {x}_{- i} ) {\vert}_{{y}_{i}}}{{Φ}_{i}^{Q} ( {y}_{- i} ) {\vert}_{{y}_{i}}} ) = {\sum }_{i} log ( \frac{{Φ}_{i}^{P} ( {x}_{- i} ) {\vert}_{{y}_{i}}}{{Φ}_{i}^{Q} ( {y}_{- i} ) {\vert}_{{y}_{i}}} ) ≦ {\sum }_{i} ( \frac{{{Φ}_{i}^{Q} ( {y}_{- i} )}_{{y}_{i}}}{{{Φ}_{i}^{P} ( {x}_{- i} )}_{{y}_{i}}} - 1 ) ≦ \frac{\sqrt{8} N}{{a}_{0}} θ$$

Combining the equations, the following can be concluded:

$${ω}_{y}^{Q} {e}^{\frac{\sqrt{8} N}{{a}_{0}} θ} ≧ Pr ( x \to y ) ≧ {ω}_{y}^{Q} {e}^{- \frac{\sqrt{8} N}{{a}_{0}} θ}$$

Now, interpreting a₀ as the probability of generating an unlikely event below the desired threshold (e.g., a “failure”), it can be noted that the probability of generating at least one such event is given by 1−(1−a₀)^{N}. Hence, if α is the pre-specified significance level, for N>>1:

$${a}_{0} \approx ( 1 - α ) / N$$

Hence, it can be concluded that at significance level ≥α, the bounds are:

$${ω}_{y}^{Q} {e}^{\frac{\sqrt{8} {N}^{2}}{z - a} θ} ≧ Pr ( x \to y ) ≧ {ω}_{y}^{Q} {e}^{- \frac{\sqrt{8} {N}^{2}}{z - a} θ}$$

This bound can be rewritten in terms of the log-likelihood of the spontaneous jump and constants independent of the initial sequence x as:

$$\vert log Pr ( x \to y ) - {C}_{0} \vert ≦ {C}_{1} θ ,$$

where the constants are given by:

$${C}_{0} = log {ω}_{y}^{Q}$$

$${C}_{1} = \frac{\sqrt{8} {N}^{2}}{1 - α}$$

As a consequence of the bounds defined above, it follows that the lower bound of the likelihood of a jump to a target sequence is higher if the final sequence is more fit in the target population. Note that the membership degree by definition quantifies the probability of generating a sequence from the inferred Qnet, and since the collection of dominant strains is far more likely when a survey of a population is conducted, it follows that the membership degree is related to the qualitative notion of fitness.

Conversely, as the fitness of the initial strain (in the neighborhood of ωₓ^{P}=1) measured by its membership degree falls, the minimum probability of going through a spontaneous jump is higher. This can be demonstrated first by noting that for x≠y:

$${ω}_{x}^{P} = 1 ⇒ Pr ( x \vert y ) = 0 ,$$

which follows because each term in the product on the right hand side is either zero or one if ωₓ^{P}=1, and there is at least one zero since x≠y. To demonstrate that the suppression of probability of a jump is not simply true if ωₓ^{P}=1 but also in the neighborhood, note that:

$${θ}_{i} ≧ \frac{1}{8} {\vert {{Φ}_{i}^{P} ( {x}_{- i} )}_{{y}_{i}} - {{Φ}_{i}^{Q} ( {y}_{- i} )}_{{y}_{i}} \vert}^{2} ⇒ {δθ}_{i} ≧ \frac{1}{4} ( {{Φ}_{i}^{P} ( {x}_{- i} )}_{{y}_{i}} - {{Φ}_{i}^{Q} ( {y}_{- i} )}_{{y}_{i}} ) {{δΦ}_{i}^{P} ( {x}_{- i} )}_{{y}_{i}} ,$$

which implies that in the neighborhood of ωₓ^{P}=1:

$$\frac{{δθ}_{i}}{{{δΦ}_{i}^{P} ( {x}_{- i} )}_{{y}_{i}}} ≧ \frac{1}{4} ( 1 - {{Φ}_{i}^{Q} ( {y}_{- i} )}_{{y}_{i}} ) > 0$$

This implies that the distance decreases as the membership degree of x falls, thus lowering the lower bound on the probability of a spontaneous jump. This is not necessarily true if x is not in the neighborhood of ωₓ^{P}=1 in the first place, and so is of lesser practical interest.

The ability of the systems and methods described herein to estimate the probability of spontaneous jump between sequences in terms of 0 has crucial implications. Embodiments of the systems and methods described herein allow for construction of a new phylogeny that directly relates the probability of jumps rather than the number of mutations between descendants, simulation of realistic trajectories in the sequence space from any given initial strain, and estimation of drift in the sequence space through analysis of the statistical characteristics of the diffusion occurring in the strain space.

### Example of Predicting Dominant Seasonal Strains of Influenza

Exemplary embodiments of the systems and methods described herein can be used to predict dominant circulating strains for the seasonal Influenza epidemics. Periodic adjustment of the Influenza vaccine components is necessary to account for antigenic drift. The flu shot is annually prepared at least six months in advance, and comprises a cocktail of historical strains determined by the WHO via global surveillance, hoping to match the circulating strain(s) in the upcoming flu season. A variety of hard-to-model effects hinders this prediction, and has limited vaccine effectiveness in recent years.

In some embodiments, analyzing the distribution of sequences using a Qnet inferred q-distance allows for seasonal drift to be estimated, which is particularly applicable to Influenza and Influenza-like viruses for which periodic adjustments of vaccine components are necessary to account for antigenic variations. The prediction of the dominant seasonal strain of Influenza is based on the following assumptions: because the probability of spontaneous jump to a strain further away in the q-distance is exponentially lower, the q-centroid of the strain distribution (the centroid computed in the q-distance metric) observed over a season is expected to move slowly, and will be close to the dominant strain in the next season. Thus, the predicted dominant strain \hat{x}^{t+1} at time t+1 as a function of the observed population at time t can be estimated as follows:

$${\hat{x}}^{ι + 1} = arg {min}_{x ∈ {P}^{t}} {\sum }_{y ∈ {P}^{t}} θ ( x , y ) ,$$

where P^{t} is the sequence population at time t. In some embodiments, the unit of time is chosen to reflect the appropriate frequency over which vaccine components are re-assessed. For the exemplary embodiment relevant to Influenza, this is typically one year. In some embodiments, this formulation can be used to test whether the Qnet predicted strain recommendations are closer to the dominant strain in the classical edit distance, when compared against the WHO vaccine recommendation for that season.

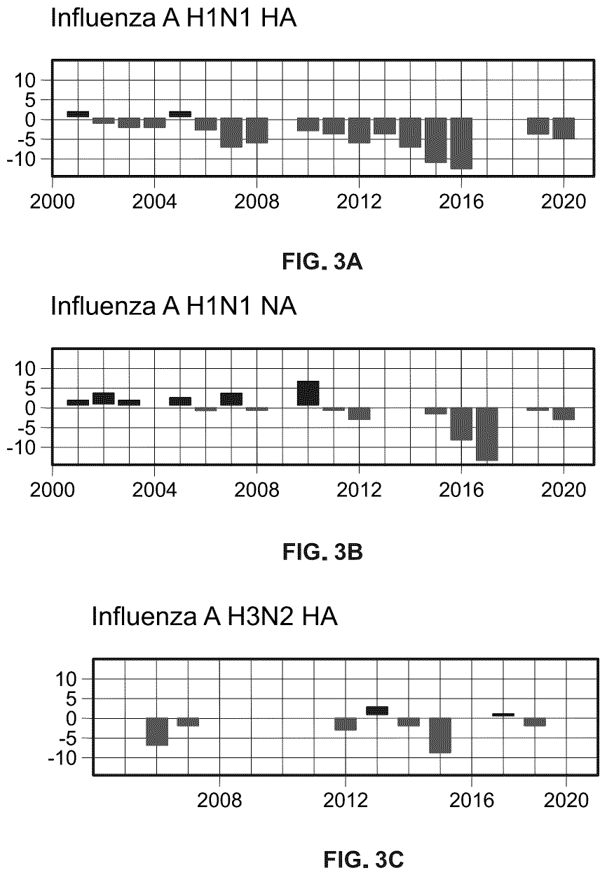

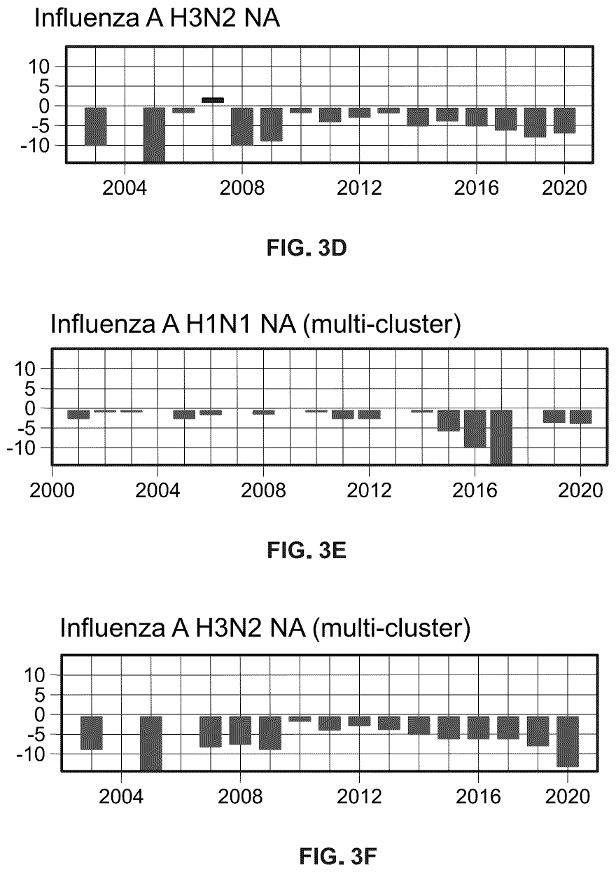

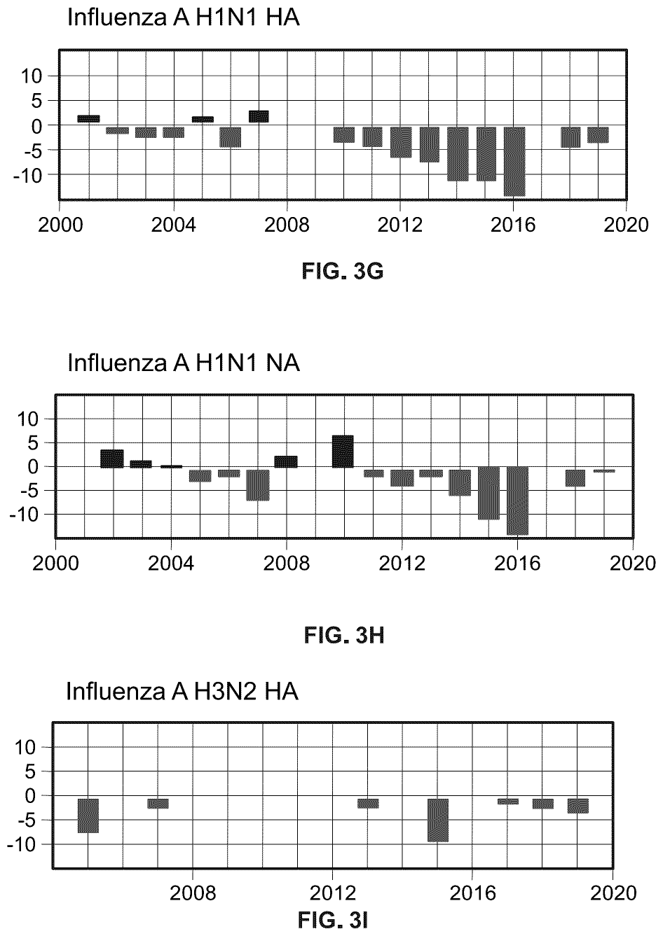

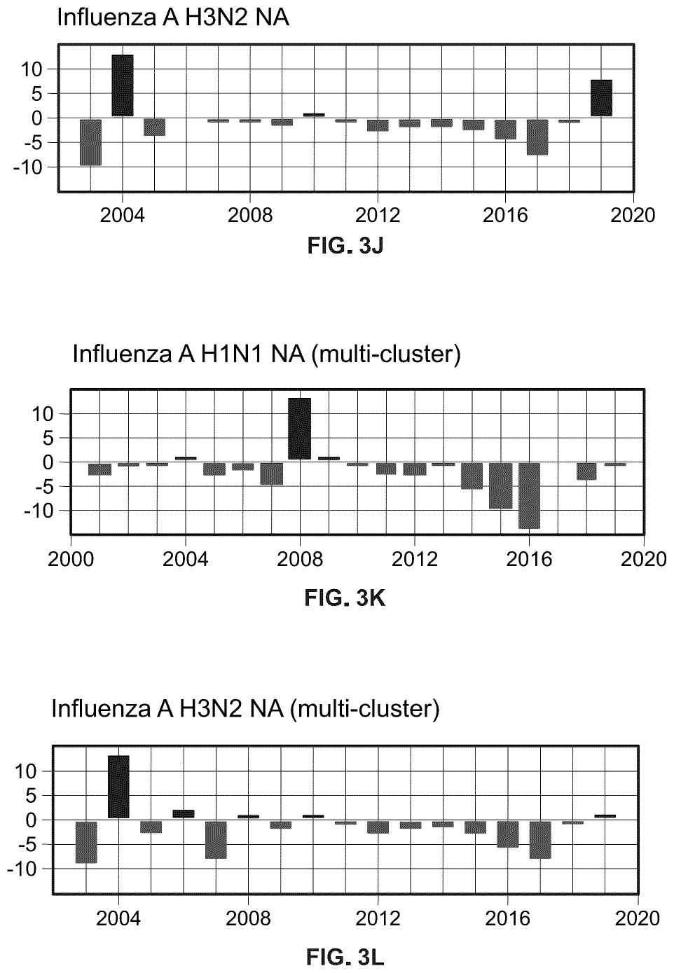

In example, the past two decades of sequence data for Influenza A (H1N1 and H3N2) were tested and the q-distance based prediction demonstrably outperformed WHO recommendations by reducing the distance between the predicted and the dominant strain (see e.g., FIG. 3A-FIG. 3L).

FIG. 3A-FIG. 3L illustrates the relative out-performance of Qnet predictions against WHO recommendations for H1N1 and H3N2 subtypes for the HA and NA coding sequences for the northern (FIG. 3A-FIG. 3F) and southern (FIG. 3G-FIG. 3L) hemispheres. The negative bars (red) indicate the reduced edit distance between the Qnet predicted sequence and the actual dominant strain that emerged that year (e.g., Qnet outperforms WHO). The positive bars (black) indicate outperformance of Qnet by WHO. Qnet outperformed WHO for the overwhelming majority of seasons. Note that the recommendations for the northern hemisphere are given in February, while those for the southern hemisphere are given at the end of December the previous year, as the flu season in the south begins a few months early. FIG. 3E-FIG. 3F and FIG. 3K-FIG. 3L show further possible improvement in NA predictions when three recommendations (e.g., multi-cluster) were returned instead of one each year.

In the example, the dominant strain was identified to be the one that occurs most frequently, computed as the centroid of the strain distribution observed in a given season in the classical sense (number of mutations). For H1N1 HA, the Qnet induced recommendation outperformed the WHO suggestion by >31% on average over the last 19 years, and >81% in the last decade in the northern hemisphere. The gains for NA over the same time periods for H1N1 for the north were >60% and >22% respectively. For the southern hemisphere, the gains for H1N1 over the last decade were >72% for HA, and >50%. The full table of results are shown in TABLE 1.

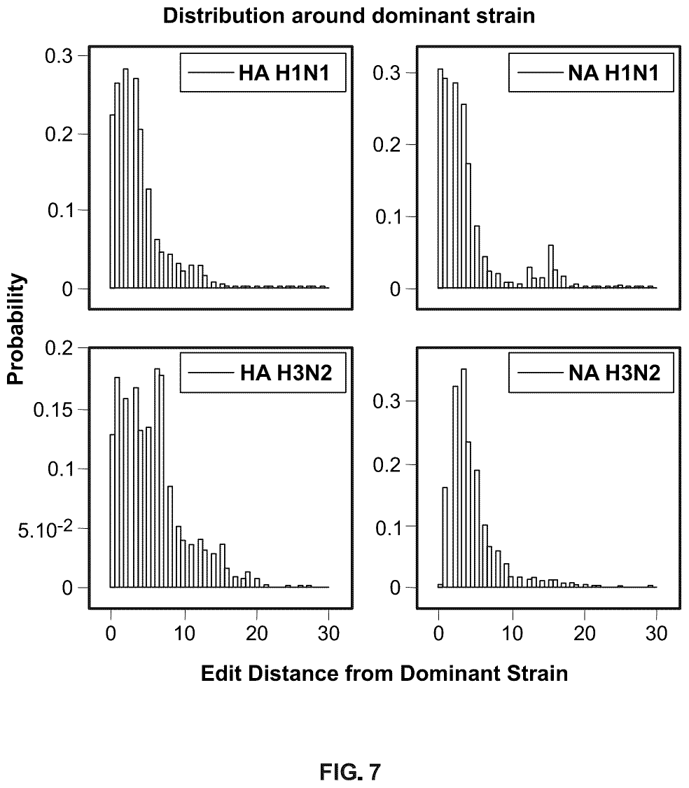

As another illustration pertaining to this implementation, FIG. 7 shows the distribution of the number of mutations from the seasonal dominant strain over the years for various strains of Influenza A. The quasispecies that circulates each season for each subtype was tightly distributed around the dominant strain on average.

FIG. 3A-FIG. 3L also illustrates the relative gains computed for both subtypes and the two hemispheres (since the flu season occupies distinct time periods and may have different dominant strains in the northern and southern hemispheres). In one implementation, additional improvement was demonstrated when multiple strains were recommended every season for the vaccine cocktail (FIG. 3F-FIG. 3L). The details of the specific strain recommendations made the Qnet approach for two subtypes (H1N1, H3N2), for two genes (HA, NA) and for the northern and the southern hemispheres over the previous 19 years are enumerated in (see e.g. TABLE 2).

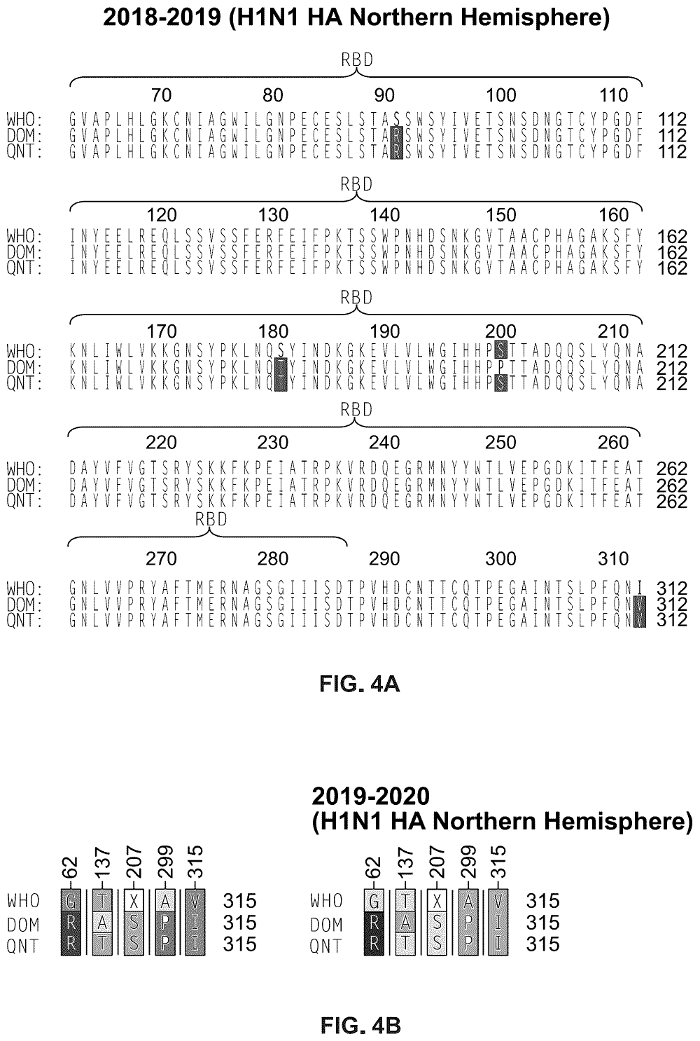

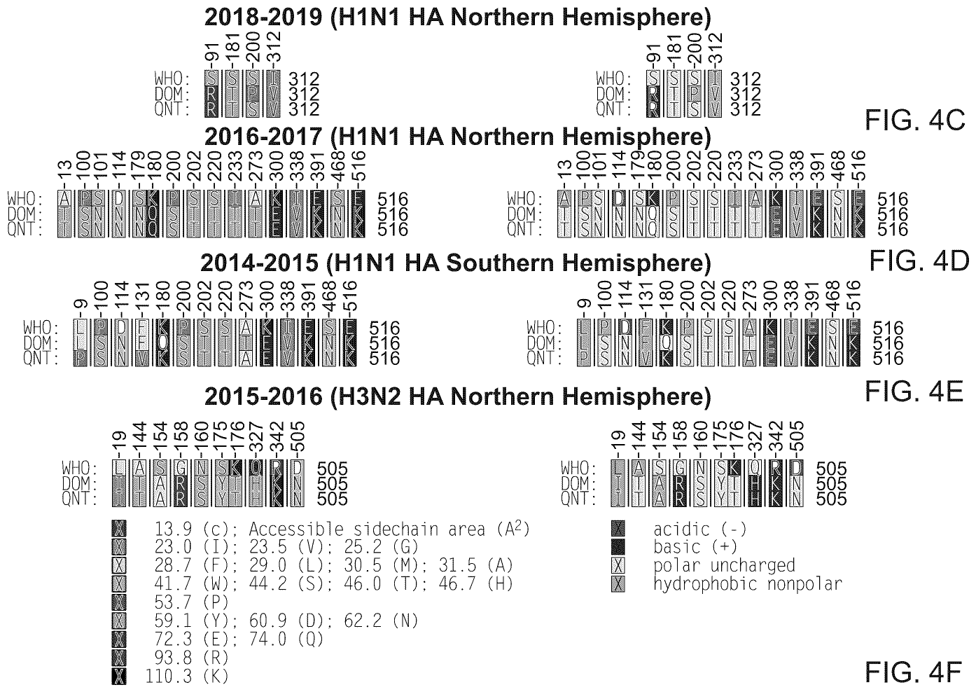

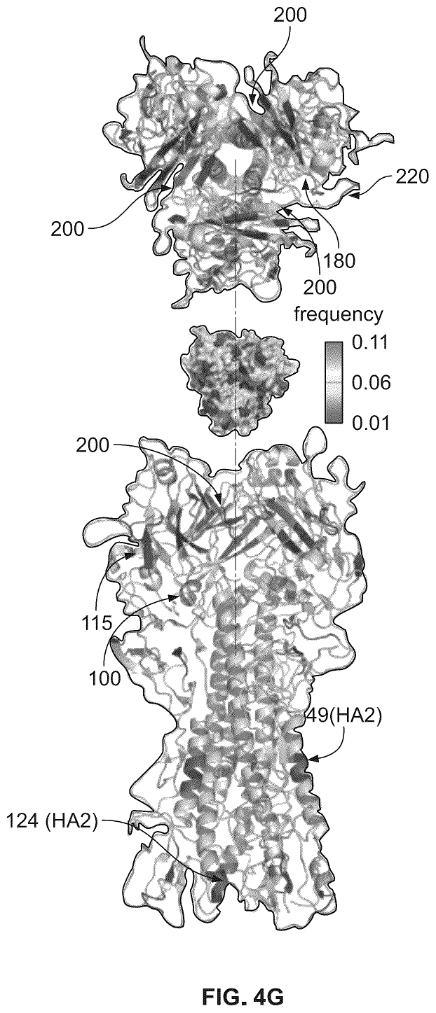

As another illustration pertaining to this implementation, FIG. 4A-FIG. 4G shows the sequence comparisons between WHO predicted, Qnet predicted, and dominant strains of influenza and a molecular model of the influenza HA protein. For the observed dominant strain, the correct Qnet deviations were localized within the receptor-binding domain (RBD), both for H1N1 and H3N2 for HA (see e.g., FIG. 4A). Additionally, by comparing the type, side chain area, and the accessible side chain area, the changes were observed to often have very different properties (see e.g., FIG. 4B-FIG. 4F). FIG. 4G shows the localization of the deviations in the molecular structure of HA, wherein the changes were most frequent in the HA1 subunit (the globular head), and around residues and structures that have been commonly implicated in receptor binding interactions, e.g. the ≈200 loop, the ≈220 loop and the ≈180-helix.

In at least one implementation, the key factors contributing to successful prediction of the dominant strain in the next season were investigated. A multivariate regression was performed with data diversity, the complexity of the inferred Qnet, and the edit distance of the WHO recommendation from the dominant strain as independent variables. Data diversity was defined as the number of clusters in the input set of sequences, such that any two sequences five or less mutations apart were in the same cluster. Qnet complexity was measured by the number of decision nodes in the component decision trees of the recursive forest. Several plausible structures of the regression equation were selected, and in each case data diversity had most important and statistically significant contribution.

### Example of Identifying Origin Species of SARS-COV-2

Embodiments of the systems and methods described herein provide for the determination of an origin host species or origin reservoir of a virus, e.g., identification of the animal host of a progenitor viral sequence. In at least one implementation, the origin species of SARS-CoV-2 was determined by quantifying the likelihood of different animal species hosting the immediate progenitor. For any novel pathogen, a plausible history of emergence can generally be constructed by estimating similarity of the consensus strain with candidates in suspected animal hosts. However, interpreting a small edit distance as being indicative of a higher chance of a species-jump is problematic, particularly if multiple potential progenitor candidates arise. In contrast, a smaller average q-distance of a novel strain from animal reservoir A vs that from B implies that there is indeed a quantifiably higher probability of a jump from A.

In some embodiments, the Qnet based phylogenetic analysis provides a significantly more reliable history of the progenitor strain. For a pandemic strain y∈H, and an animal strain x∈P:

$$log \frac{1}{{ω}_{y}} Pr ( x \to y ) ≧ - \frac{\sqrt{8} {N}^{2}}{1 - α} θ ( x , y ) ⇒ log \frac{1}{{ω}_{y}} {E}_{x ∈ P} Pr ( x \to y ) ≧ - \frac{\sqrt{8} {N}^{2}}{1 - α} {E}_{x ∈ P} θ ( x , y )$$

There are constants C, C′ such that

$$- log {E}_{x ∈ P} Pr ( x \to y ) ≦ C + {C}^{′} {E}_{x ∈ P} θ ( x , y )$$

Note that because N is known, C′ can be calculated without the knowledge of the pandemic strain y. For the example of the SARS-CoV-2 spike protein, at 95% significance:

$${C}^{′} = {3187}^{2} \times 1 / ( 1 - 0.95 ) \times \sqrt{8} = 5.75 \times {10}^{8}$$

If the pandemic strain is known and it is desired to compare and contrast the likelihood of jump from potential hosts after the emergence event, then C can be explicitly calculated. For the example of SARS-CoV-2, this estimate was calculated as 4,805.4 (see e.g., FIG. 10A), which leads to the following linear relationship between log-likelihood of emergence and the average distance calculated in the Qnet framework:

$$- log {E}_{x ∈ P} Pr ( x \to y ) ≦ 4.8054 \times {10}^{3} + 5.75 \times {10}^{8} {E}_{x ∈ P} θ ( x , y ) ,$$

thus providing a quantitative ranking of potential progenitor hosts. It follows that for rank-ordering potential hosts, only the average distance E_{x∈P}θ(x, y) needs to be considered. It also follows from the relative magnitudes of the constants in the case of SARS-CoV-2, that C can be ignored, yielding the approximation:

$$log {E}_{x ∈ P} Pr ( x \to y ) ≧ - 5.75 \times {10}^{8} {E}_{x ∈ P} θ ( x , y )$$

For the example of SARS-CoV-2, the fitness term is approximately five orders of magnitude smaller, which implies the jump probabilities are roughly symmetric. However, this is not required to be true in general. At the same time, it is important to note that in some embodiments the probability of jump from strain x to strain y vs. the reverse is actually asymmetric due to the contribution from the population-specific membership degree.

The numerical bounds were estimated on the likelihood of the SARS-CoV-2 progenitor arising from specific hosts.

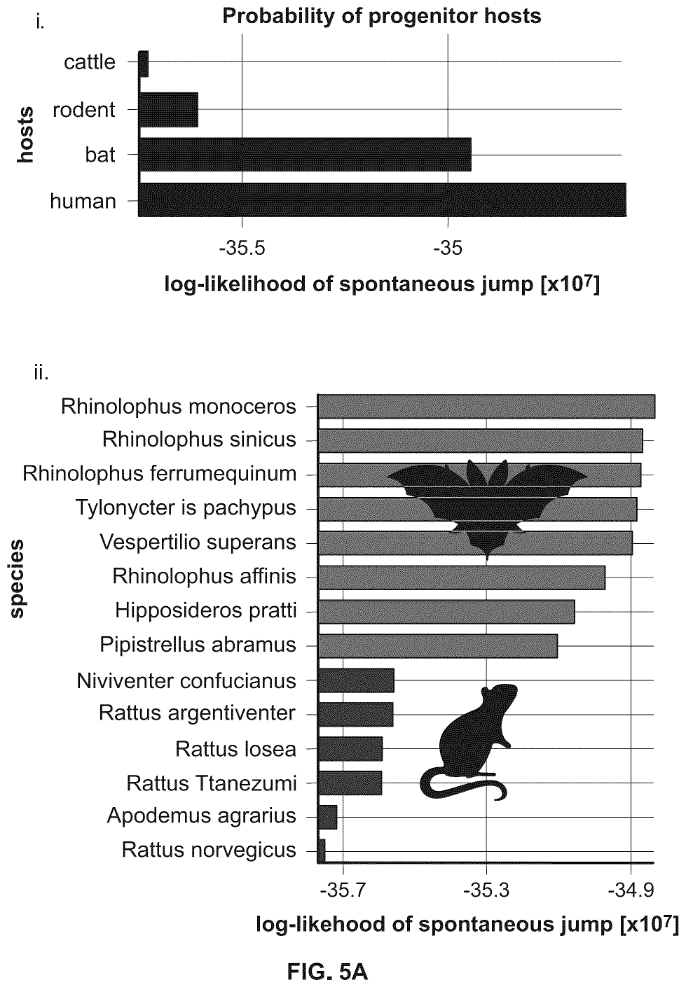

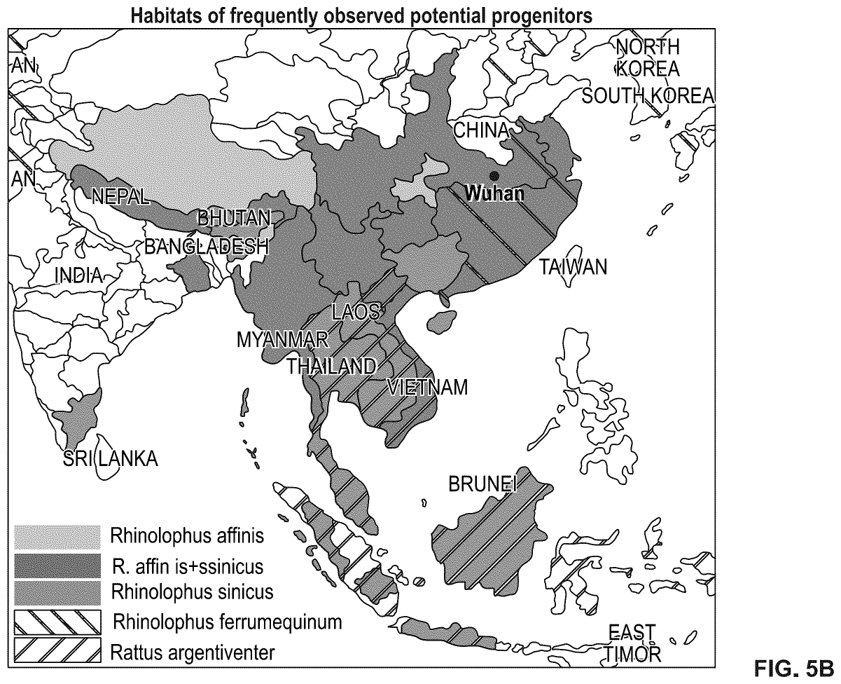

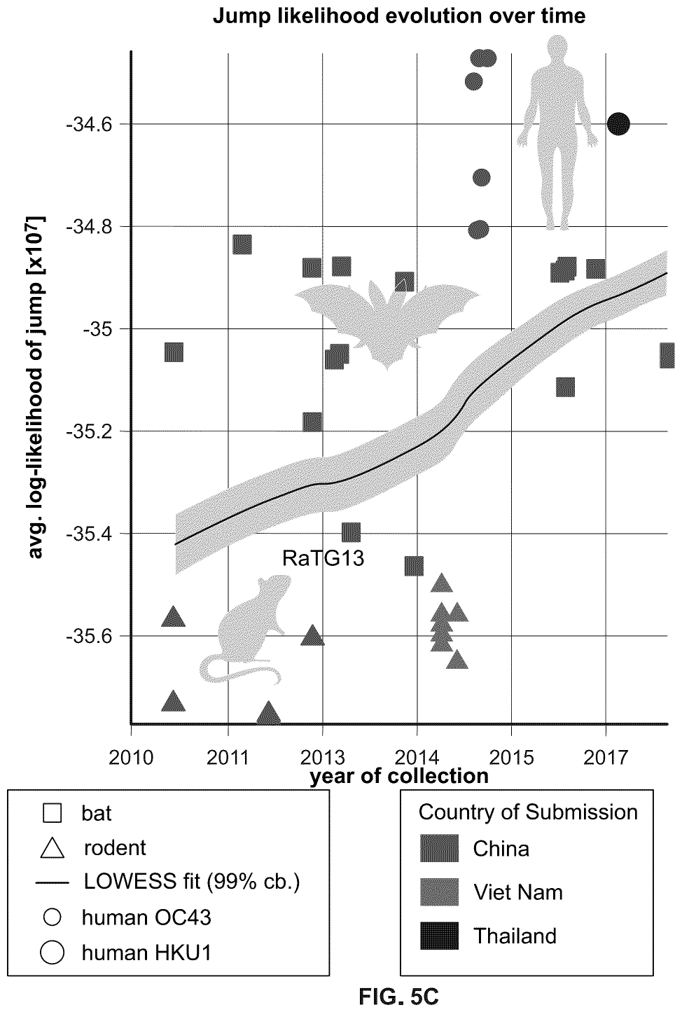

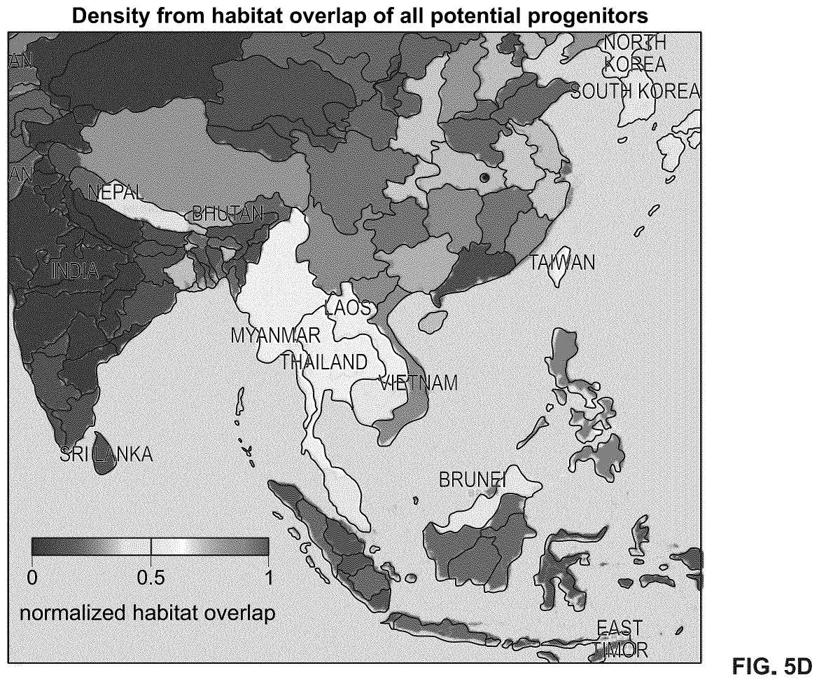

FIG. 5A-FIG. 5D shows the prediction of animal hosts for likely progenitors of SARS-CoV-2. FIG. 5A (i) shows the average lower bounds on the log-likelihood of jump from different animal hosts to the set of SARS-CoV-2 sequences collected in the early days of the pandemic. FIG. 5A (ii) shows the lower bounds on the log-likelihood of jump from specific species to their respective nearest SARS-CoV-2 neighbors (among sequences collected in the early days of the pandemic). FIG. 5B shows the geographic extent of the habitats of the top four most frequently occurring species among the list shown in FIG. 5A (ii). Also, the location of Wuhan, China, ground zero for COVID19 is shown. FIG. 5C plots the lower bound on log-likelihood of various sequences to their nearest neighbors over the time of collection, suggesting a trend of increasing risk over time, and across hosts, as evidenced by a nearly constant gradient LOWESS fit (black line) with 99% confidence bounds. FIG. 5D shows the normalized footprint of risk-mediating hosts from overlapping the geographic extents of the habitats of all species from the list in FIG. 5A (ii).

Betacoronavirus sequences from NCBI databases corresponding to different animal hosts were used to estimate the mean q-distance of SARS-CoV-2 sequences to bats, mouse/rodents, cattle (including camels) and pre-existing human strains including SARS-CoV1, OC43 and HKU1 strains (see e.g., FIG. 5A, showing the average log-likelihood of jump from different animal species). No a priori restriction was used to hosts geographically bound to South East Asia, and it was demonstrated that this localization arises naturally from the analysis. The results corroborate other studies suggesting high probability of the progenitor originating from bats. (see e.g., FIG. 5A (i), which shows the average lower bound of the log-likelihood of a spontaneous jump from broad host categories to SARS-CoV-2 strains collected up to early March in 2020).

A ranked list of related bat species with the highest potential of hosting a SARS-CoV-2 progenitor was also identified (see e.g., FIG. 5A (ii), which shows the minimum likelihood of jump to the nearest SARS-CoV-2 strain for the respective host species). Additionally, a high likelihood of a close ancestor of SARS-CoV-2 existing in rodents was also discovered (see e.g., FIG. 5A).

### Example of Constructing Qnet Phylogenetic Trees (Q-Phylogeny)

In some embodiments, the systems and methods described herein can be used to construct phylogenetic trees based on the Qnet provided metric, q-distance. The majority of algorithms for constructing phylogenies generally require a notion of distance between biological sequences, and the edit distance is the one that is most commonly used. In some embodiments, the Qnet induced distance or q-distance is used to construct phylogenetic trees that are distinct from those obtained using the classical metric of edit distance. In some embodiments, the Qnet induced phylogeny (e.g., Q-phylogeny) is reflective of evolutionary change in a manner that conventional trees are not. As a path is traced in a Q-phylogeny, the probability of the changes represented by that path can be explicitly computed. This probability can be bounded above and below by a function of the total path length, e.g., the sum of the q-distances along the path. As an example, for the path

$$x - {x}^{0} \to ⋯ {x}^{k} \to ⋯ {x}^{m} = z , \\ \frac{\sqrt{8} {N}^{2}}{1 - α} Θ ≧ log Pr ( x \to z ) - {\sum }_{i = 1}^{m} log {ω}_{{x}^{i}} ≧ \frac{\sqrt{8} {N}^{2}}{1 - α} Θ , \\ where Θ = {\sum }_{i = 1}^{m} θ ( {x}^{k - 1} , {x}^{k} )$$

Considering only the lower bound,

$$log \frac{Pr ( x \to z )}{{\prod }_{i = 1}^{m} {ω}_{{x}^{i}}} ≧ - \frac{\sqrt{8} {N}^{2}}{1 - α} Θ$$

where ωₓi is the membership probability in the base population of the strain xⁱ. Thus, closer phylogenetic distance can be related to explicit probability of spontaneous jump. The definition of the distance function in the Qnet framework allows the summation in the equation, allowing the use of standard tools to construct the phylogenetic tree in some embodiments.

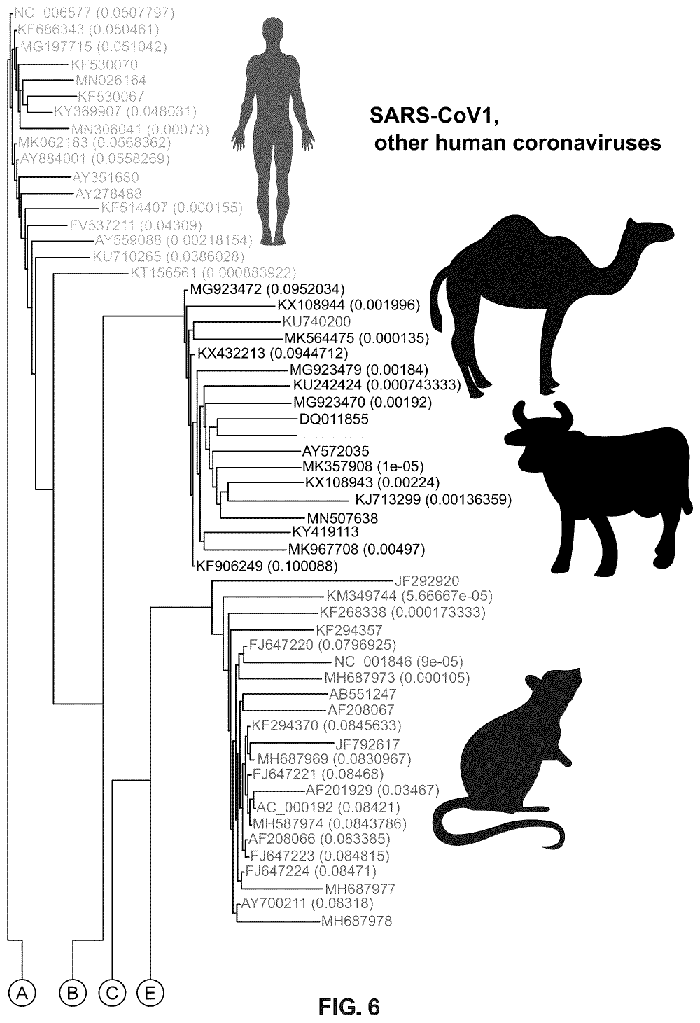

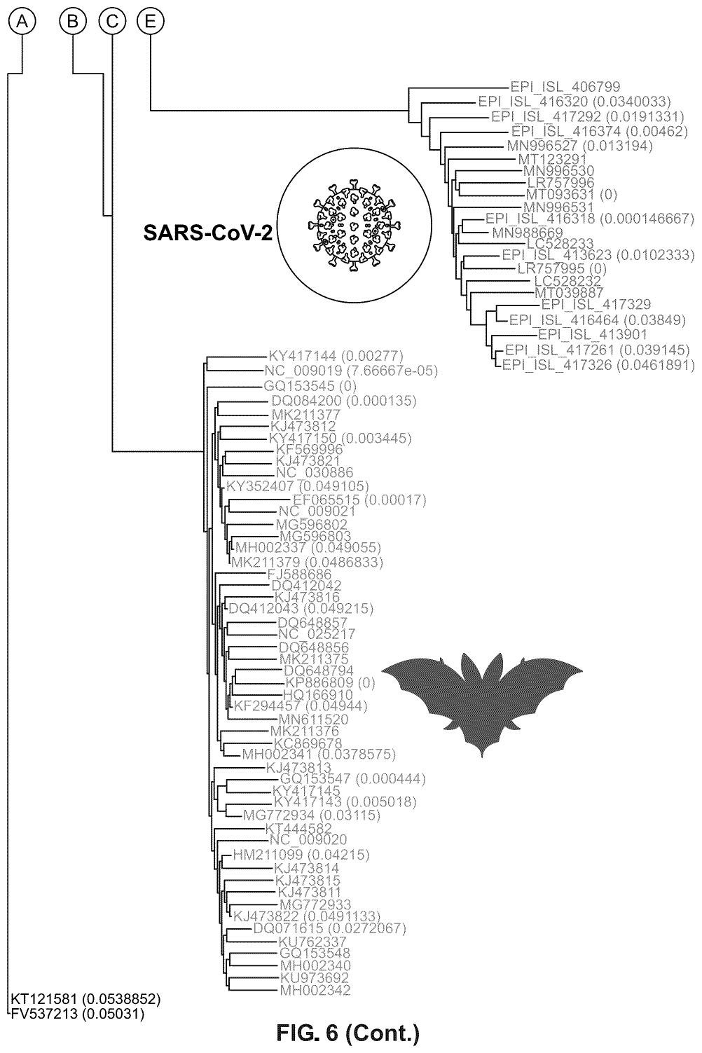

FIG. 6 illustrates an example of a q-distance induced phylogenetic tree. Importantly, the chronology of SARS-CoV-2 vs. existing betacoronaviruses was automatically preserved, as well as an intriguing clade-hierarchy between bat, rodent and SARS-CoV-2 strains. Some branches of the phylogenetic tree were collapsed, and the numbers in bracket list the magnitude of q-distance within which leaves were collapsed.

The q-distance induced phylogenetic tree illustrates a previously unknown role of rodents in the SARS-CoV-2 pandemic: SARS-CoV-2 strains and betacoronaviruses from rodents appeared in the same clade nested within the clade comprising betacoronaviruses from bats, rodents and SARS-CoV-2 strains (while the rodent strains were not actually closer than those isolated in bats, see e.g., FIG. 6).

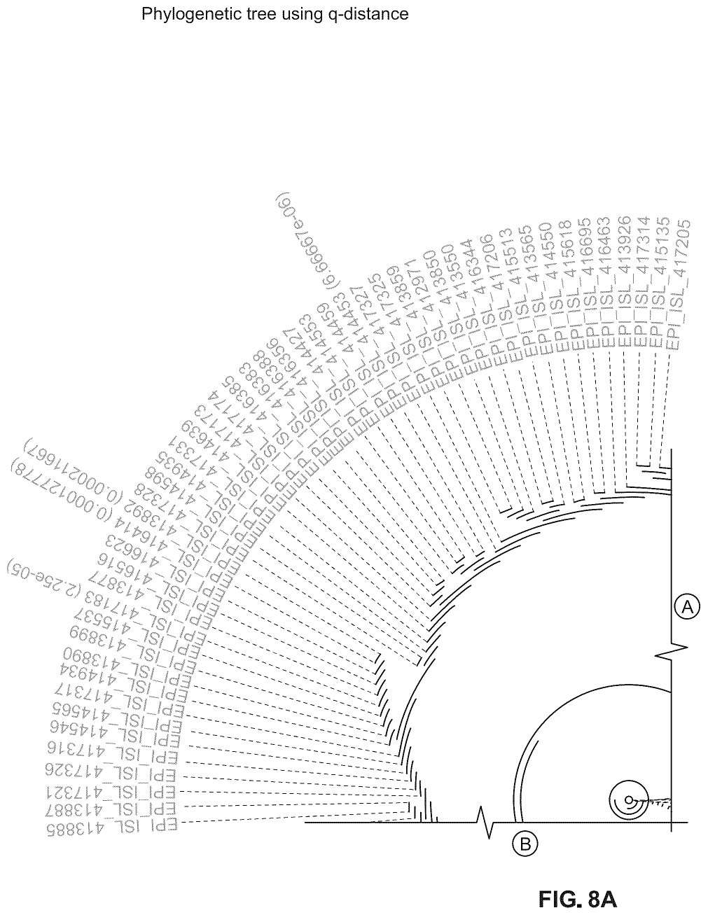

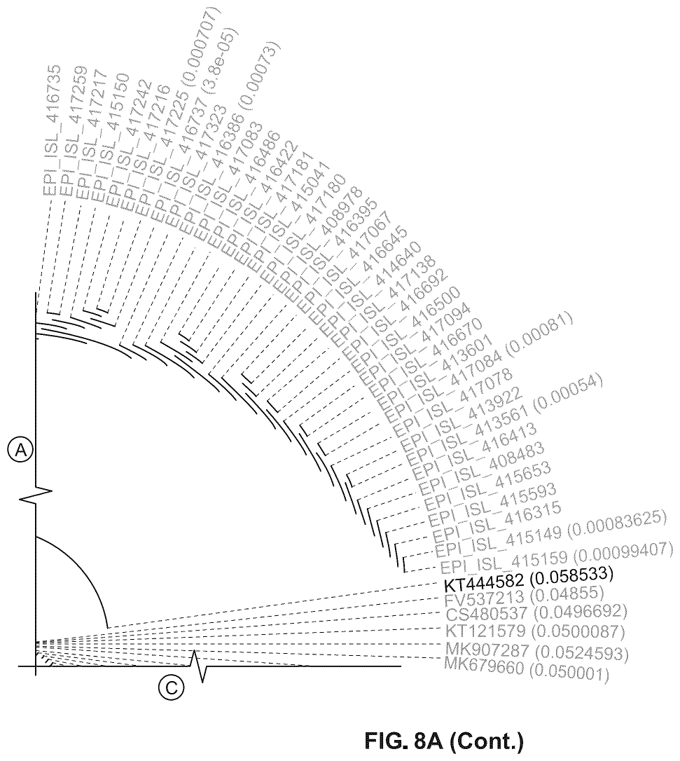

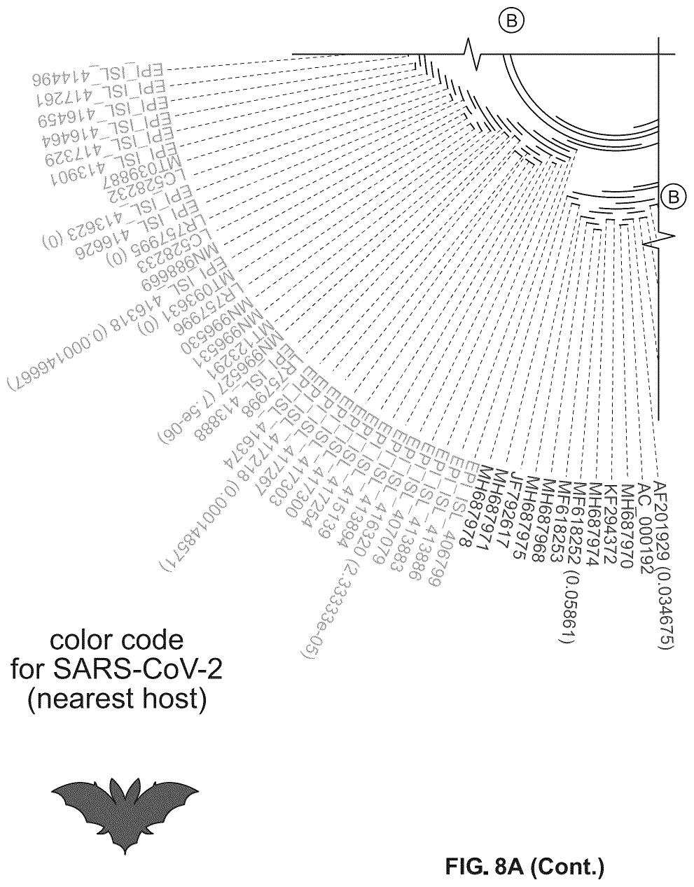

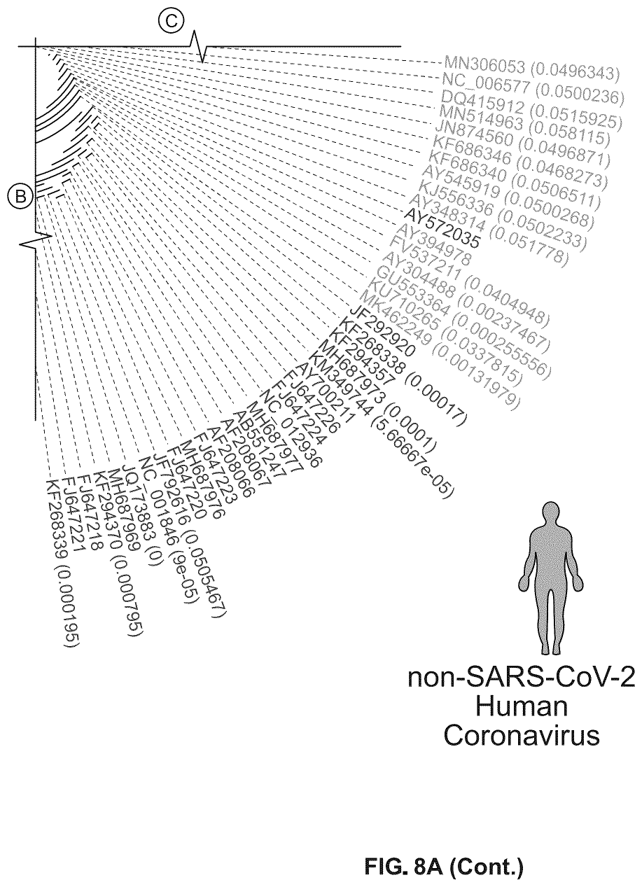

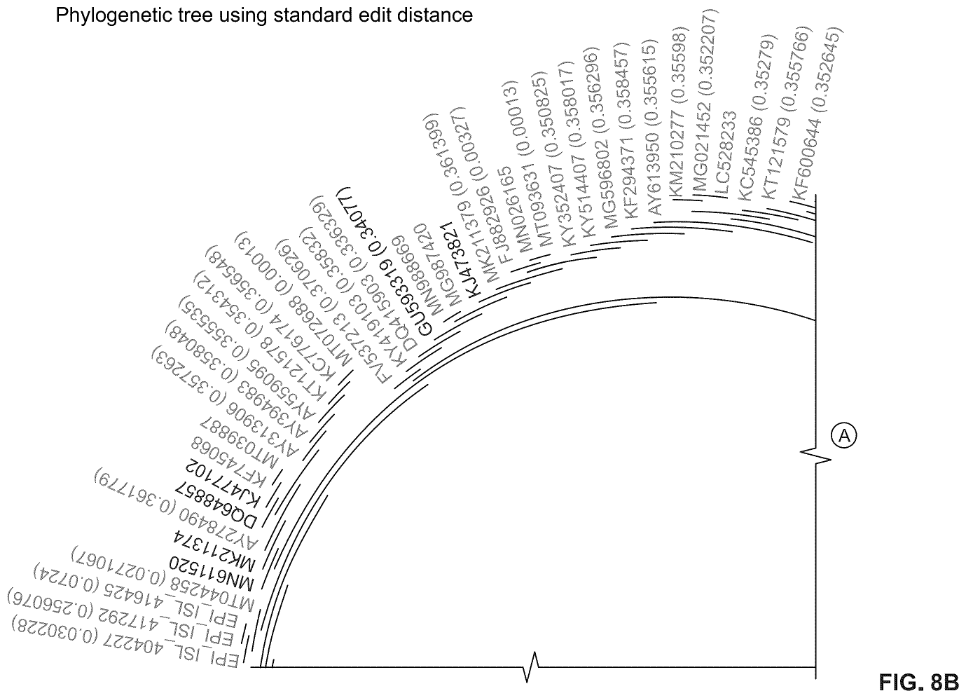

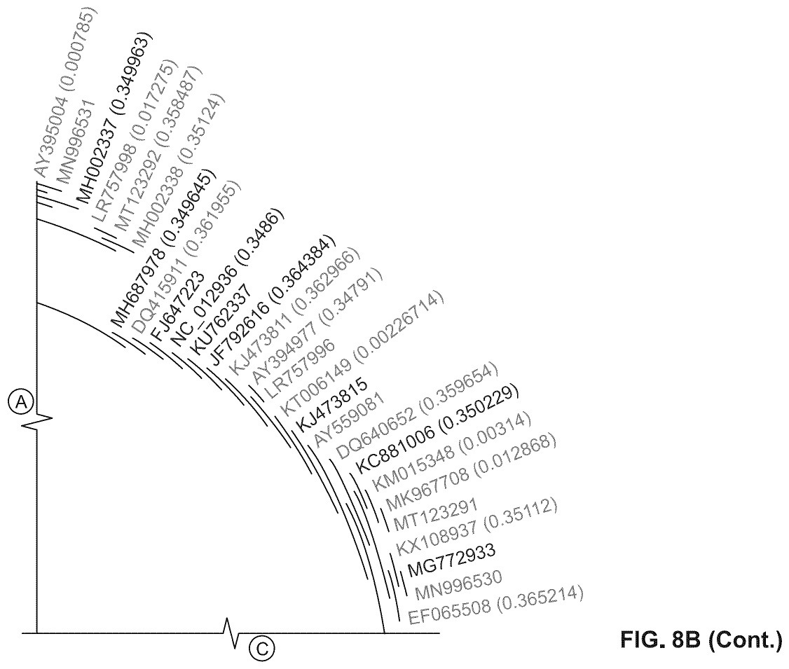

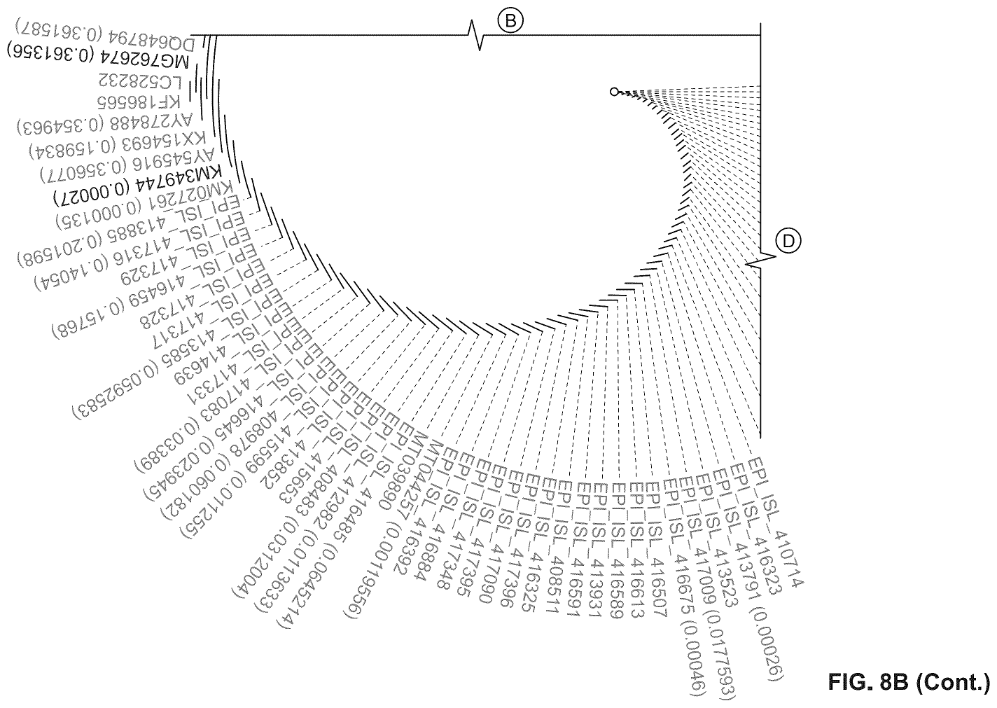

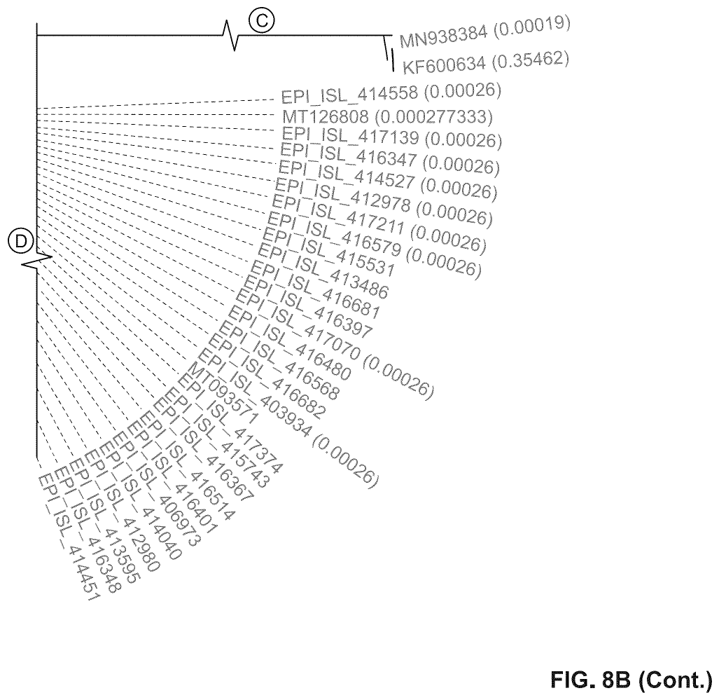

FIG. 8A-FIG. 8B also illustrates examples of phylogenetic trees derived from q-distance (FIG. 8A) and classical edit distance (FIG. 8B). The numbers within brackets are the distance within which the specific branch is collapsed for visualization. The classical edit distance produced a phylogeny which clearly violates chronological ordering, as the novel coronavirus appears before strains that have been collected years before, including the SARs-1 strains. The new distance using Qnet automatically respected this known ordering.

### Example of Validating Qnet Constraints in Silico

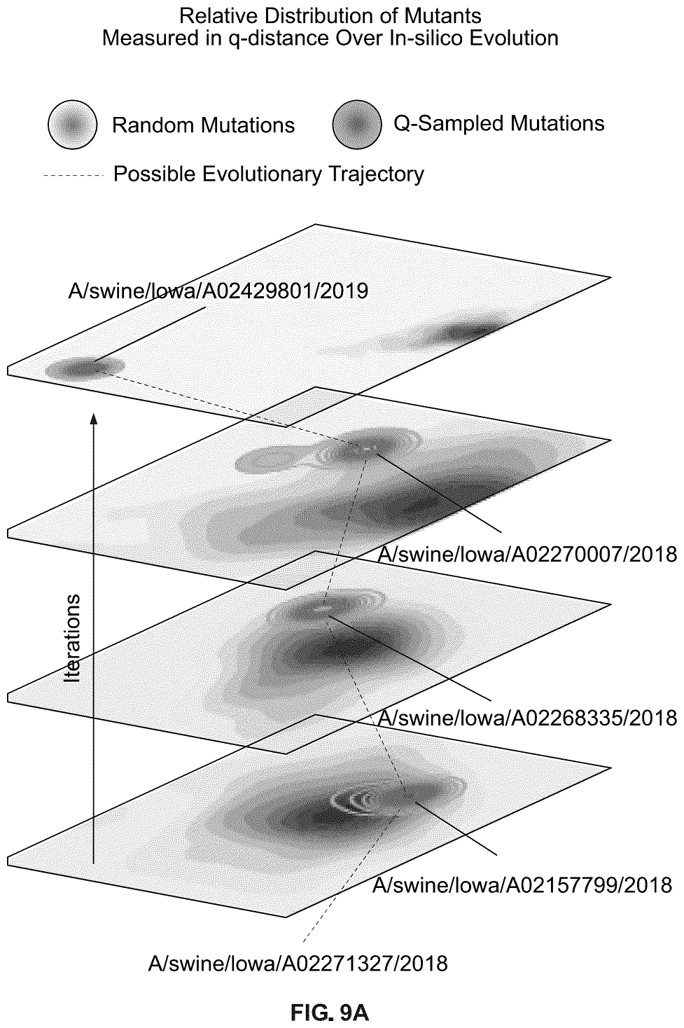

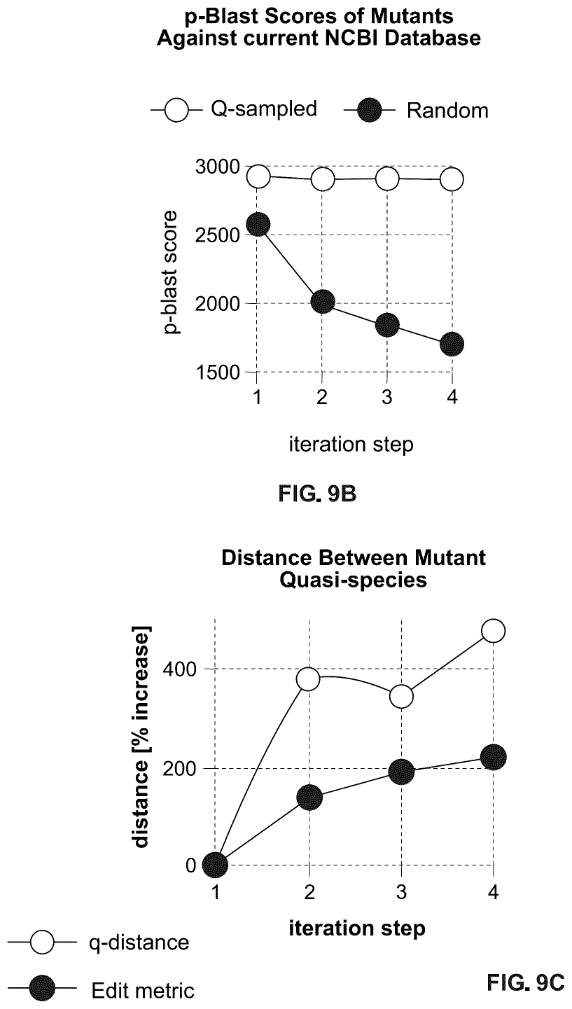

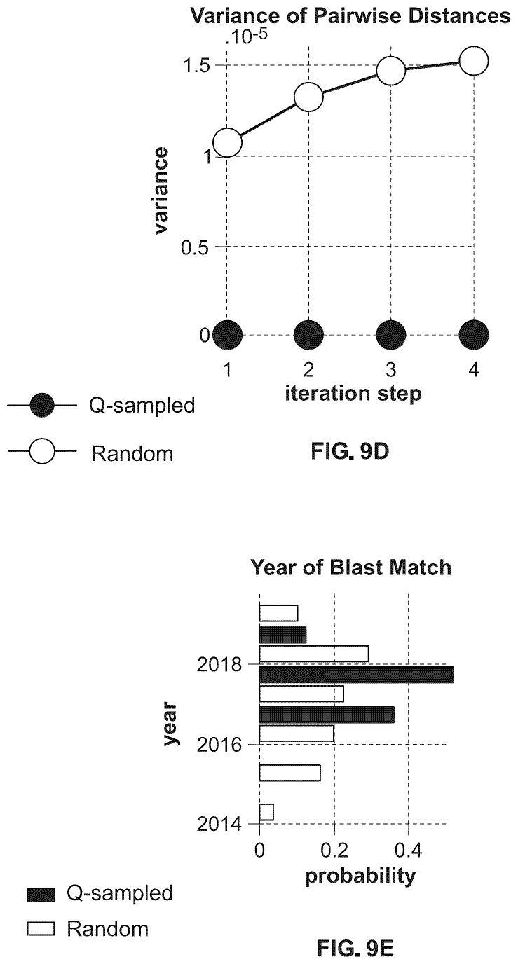

In silico corroboration of the Qnet constraints was performed to corroborate that the constraints represented within an inferred Qnet are indeed reflective of the biology at play. The results of simulated mutational perturbations were compared to sequences from databases (for which Qnets were already constructed), and then the NCBI BLAST tool was used to identify if the perturbed sequences match with existing sequences in the databases (and if so, then where and how many matches they produce). FIG. 9A-FIG. 9E illustrates the results comparing such Qnet constrained perturbations against random variations.

FIG. 9A-FIG. 9E demonstrates the validation of q-distance in silico using Influenza A sequences from the NCBI database. FIG. 9A illustrates that the Qnet-induced modeling of evolutionary trajectories initiated from known haemagluttinin (HA) sequences are distinct from random paths in the strain space. In particular, random trajectories have more variance, and more importantly, diverge to different regions of the landscape compared to Qnet predictions. FIG. 9B-FIG. 9E show that unconstrained Q-sampling produces sequences that maintain a higher degree of similarity to known sequences, as verified by blasting against known HA sequences, have a smaller rate of growth of variance, and produce matches in closer time frames to the initial sequence. FIG. 9C shows that this is not due to simply restricting the mutational variations, which increase rapidly in both the Qnet and the classical metric.

In at least one implementation, the systems and methods described herein demonstrate that in contrast to random variations, which rapidly diverge the trajectories, the Qnet constraints produced smaller variance in the trajectories, maintained a high degree of match as trajectories are extended, and produced matches closer in time to the collection time of the initial sequence-suggesting that the Qnet does indeed capture realistic constraints.

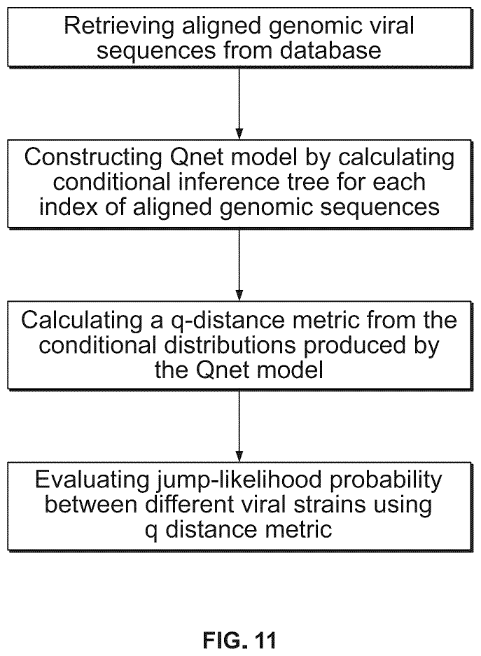

FIG. 11 is an example method of predicting the likelihood that a viral sequence will mutate into another according to the techniques describe above. In the method, aligned genomic viral sequences are acquired from a database. A Qnet model is constructed by calculating the conditional inference tree for each index of the aligned genomic sequences. A q-distance metric is calculated from the conditional distributions produced by the Qnet model. The jump-likelihood probability between different viral strains can then be evaluated using the q-distance metric.

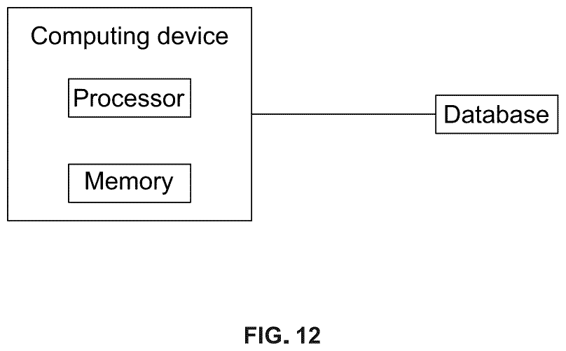

FIG. 12 is an example system for evaluating the jump-likelihood probability between different viral strains. The system may be used, for example, to perform the method shown in FIG. 11. The system includes a computing device and a database. The computing device is communicatively coupled to the database to receive data from the database. In some embodiments, the computing retrieves aligned sequence data from the database. Moreover, in some embodiments, the database is integrated into the computing device, while in other embodiments, the database is located remote from the computing device.

The computing device may include, a general purpose central processing unit (CPU), a microcontroller, a reduced instruction set computer (RISC) processor, an application specific integrated circuit (ASIC), a programmable logic circuit (PLC), and/or any other circuit or processor capable of executing the functions described herein. The methods described herein may be encoded as executable instructions embodied in a computer-readable medium including, without limitation, a storage device and/or a memory device. Such instructions, when executed by a processor, cause the processor to perform at least a portion of the methods described herein.

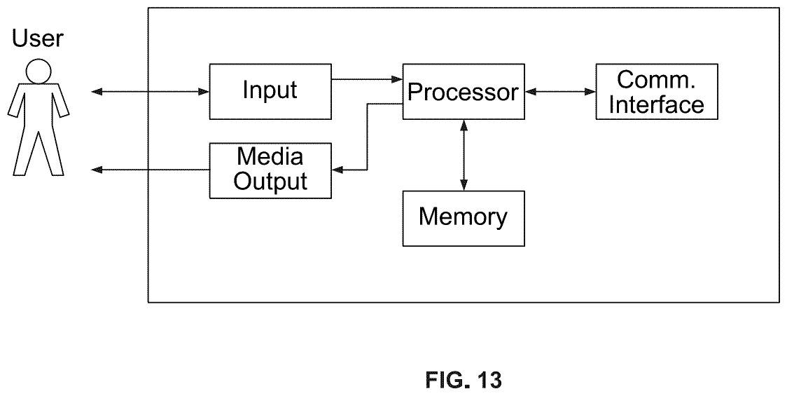

FIG. 13 is an example computing device for use as the computing device shown in FIG. 12. The computing device includes a processor, a memory, a media output component, an input device, and a communications interface. Other embodiments include different components, additional components, and/or do not include all components shown in FIG. 13.

The computing device may include, a general purpose central processing unit (CPU), a microcontroller, a reduced instruction set computer (RISC) processor, an application specific integrated circuit (ASIC), a programmable logic circuit (PLC), and/or any other circuit or processor capable of executing the functions described herein. The methods described herein may be encoded as executable instructions embodied in a computer-readable medium including, without limitation, a storage device and/or a memory device. Such instructions, when executed by a processor, cause the processor to perform at least a portion of the methods described herein.

The processor is configured for executing instructions. In some embodiments, executable instructions are stored in the memory. The processor may include one or more processing units (e.g., in a multi-core configuration). The term processor, as used herein, refers to central processing units, microprocessors, microcontrollers, reduced instruction set circuits (RISC), application specific integrated circuits (ASIC), logic circuits, and any other circuit or processor capable of executing the functions described herein. The above are examples only, and are thus not intended to limit in any way the definition and/or meaning of the term “processor.”

The media output component is configured for presenting information to the user (e.g., the operator of the system). The media output component is any component capable of conveying information to the user. In some embodiments, the media output component includes an output adapter such as a video adapter and/or an audio adapter. The output adapter is operatively connected to the processor and operatively connectable to an output device such as a display device (e.g., a liquid crystal display (LCD), light emitting diode (LED) display, organic light emitting diode (OLED) display, cathode ray tube (CRT), “electronic ink” display, one or more light emitting diodes (LEDs)) or an audio output device (e.g., a speaker or headphones).

The computing device includes, or is connected to, the input device for receiving input from the user. The input device is any device that permits the computing device to receive analog and/or digital commands, instructions, or other inputs from the user, including visual, audio, touch, button presses, stylus taps, etc. The input device may include, for example, a variable resistor, an input dial, a keyboard/keypad, a pointing device, a mouse, a stylus, a touch sensitive panel (e.g., a touch pad or a touch screen), a gyroscope, an accelerometer, a position detector, an audio input device, or any combination thereof. A single component such as a touch screen may function as both an output device of the media output component and the input device.

The memory stores computer-readable instructions for performance of the techniques described herein. In some embodiments, the memory stores computer-readable instructions for providing a user interface to the user via media output component and, receiving and processing input from input device. The memory may include, but is not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). Although illustrated as separate from the processor, in some embodiments the memory is combined with the processor, such as in a microcontroller or microprocessor, but may still be referred to separately. The above memory types are example only, and are thus not limiting as to the types of memory usable for storage of a computer program.

The communication interface enables the computing device to communicate with remote devices and systems, such as remote databases, remote computing devices, and the like, and may include more than one communication interface for interacting with more than one remote device or system. The communication interfaces may be wired or wireless communications interfaces that permit the computing device to communicate with the remote devices and systems directly or via a network. Wireless communication interfaces may include a radio frequency (RF) transceiver, a Bluetooth® adapter, a Wi-Fi transceiver, a ZigBee® transceiver, a near field communication (NFC) transceiver, an infrared (IR) transceiver, and/or any other device and communication protocol for wireless communication. (Bluetooth is a registered trademark of Bluetooth Special Interest Group of Kirkland, Washington: ZigBee is a registered trademark of the ZigBee Alliance of San Ramon, California.) Wired communication interfaces may use any suitable wired communication protocol for direct communication including, without limitation, USB, RS232, I2C, SPI, analog, and proprietary I/O protocols. In some embodiments, the wired communication interfaces include a wired network adapter allowing the computing device to be coupled to a network, such as the Internet, a local area network (LAN), a wide area network (WAN), a mesh network, and/or any other network to communicate with remote devices and systems via the network.

The computer systems discussed herein may include additional, less, or alternate functionality, including that discussed elsewhere herein. The computer systems discussed herein may include or be implemented via computer-executable instructions stored on non-transitory computer-readable media or medium.

A processor or a processing element may employ artificial intelligence and/or be trained using supervised or unsupervised machine learning, and the machine learning program may employ a neural network, which may be a convolutional neural network, a deep learning neural network, or a combined learning module or program that learns in two or more fields or areas of interest. Machine learning may involve identifying and recognizing patterns in existing data in order to facilitate making predictions for subsequent data. Models may be created based upon example inputs in order to make valid and reliable predictions for novel inputs.

In some aspects, at least one of a plurality of machine learning methods and algorithms may be applied, which may include but are not limited to: linear or logistic regression, instance-based algorithms, regularization algorithms, decision trees, Bayesian networks, cluster analysis, association rule learning, artificial neural networks, deep learning, dimensionality reduction, and support vector machines. In various aspects, the implemented machine learning methods and algorithms are directed toward at least one of a plurality of categorizations of machine learning, such as supervised learning, unsupervised learning, and reinforcement learning.

In one aspect, machine learning methods and algorithms are directed toward supervised learning, which involves identifying patterns in existing data to make predictions about subsequently received data. Specifically, machine learning methods and algorithms directed toward supervised learning are “trained” through training data, which includes example inputs and associated example outputs. Based on the training data, the machine learning methods and algorithms may generate a predictive function which maps outputs to inputs and utilize the predictive function to generate machine learning outputs based on data inputs.

In another aspect, machine learning methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or machine learning outputs, is organized according to an algorithm-determined relationship.

In yet another aspect, machine learning methods and algorithms are directed toward reinforcement learning, which involves optimizing outputs based on feedback from a reward signal. Specifically, machine learning methods and algorithms directed toward reinforcement learning may receive a user-defined reward signal definition, receive a data input, utilize a decision-making model to generate a machine learning output based on the data input, receive a reward signal based on the reward signal definition and the machine learning output, and alter the decision-making model so as to receive a stronger reward signal for subsequently generated machine learning outputs.

As will be appreciated based upon the foregoing specification, the above-described embodiments of the disclosure may be implemented using computer programming or engineering techniques including computer software, firmware, hardware or any combination or subset thereof. Any such resulting program, having computer-readable code means, may be embodied or provided within one or more computer-readable media, thereby making a computer program product, i.e., an article of manufacture, according to the discussed embodiments of the disclosure. The computer-readable media may be, for example, but is not limited to, a fixed (hard) drive, diskette, optical disk, magnetic tape, semiconductor memory such as read-only memory (ROM), and/or any transmitting/receiving medium, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are example only, and are thus not intended to limit in any way the definition and/or meaning of the term “processor.”

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are example only, and are thus not limiting as to the types of memory usable for storage of a computer program.

In another embodiment, a computer program is provided, and the program is embodied on a computer-readable medium. In an example embodiment, the system is executed on a single computer system, without requiring a connection to a server computer. In a further example embodiment, the system is being run in a Windows® environment (Windows is a registered trademark of Microsoft Corporation,

Redmond, Washington). In yet another embodiment, the system is run on a mainframe environment and a UNIX® server environment (UNIX is a registered trademark of X/Open Company Limited located in Reading, Berkshire, United Kingdom). In a further embodiment, the system is run on an iOS® environment (iOS is a registered trademark of Cisco Systems, Inc. located in San Jose, CA). In yet a further embodiment, the system is run on a Mac OS® environment (Mac OS is a registered trademark of Apple Inc. located in Cupertino, CA). In still yet a further embodiment, the system is run on Android® OS (Android is a registered trademark of Google, Inc. of Mountain View, CA). In another embodiment, the system is run on Linux® OS (Linux is a registered trademark of Linus Torvalds of Boston, MA). The application is flexible and designed to run in various different environments without compromising any major functionality.

In some embodiments, the system includes multiple components distributed among a plurality of computer devices. One or more components may be in the form of computer-executable instructions embodied in a computer-readable medium. The systems and processes are not limited to the specific embodiments described herein. In addition, components of each system and each process can be practiced independent and separate from other components and processes described herein. Each component and process can also be used in combination with other assembly packages and processes. The present embodiments may enhance the functionality and functioning of computers and/or computer systems.

Any logic flows depicted in the figures do not require the particular order shown, or sequential order, to achieve desirable results. In addition, other steps may be provided, or steps may be eliminated, from the described flows, and other components may be added to, or removed from, the described systems. Accordingly, other embodiments are within the scope of the following claims.

It will be appreciated that the above embodiments that have been described in particular detail are merely example or possible embodiments, and that there are many other combinations, additions, or alternatives that may be included.

Also, the particular naming of the components, capitalization of terms, the attributes, data structures, or any other programming or structural aspect is not mandatory or significant, and the mechanisms that implement the disclosure or its features may have different names, formats, or protocols. Further, the system may be implemented via a combination of hardware and software, as described, or entirely in hardware elements. Also, the particular division of functionality between the various system components described herein is merely one example, and not mandatory: functions performed by a single system component may instead be performed by multiple components, and functions performed by multiple components may instead performed by a single component.

Approximating language, as used herein throughout the specification and claims, may be applied to modify any quantitative representation that could permissibly vary without resulting in a change in the basic function to which it is related. Accordingly, a value modified by a term or terms, such as “about” and “substantially”, are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

Various changes, modifications, and alterations in the teachings of the present disclosure may be contemplated by those skilled in the art without departing from the intended spirit and scope thereof. It is intended that the present disclosure encompass such changes and modifications.

This written description uses examples to describe the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

### Tables

TABLE 1
Out-performance of Qnet recommendations over
WHO for Influenza A vaccine composition

| | | | Two decades (% | One decade (% |
|---|---|---|---|---|
| subtype | gene | hemisphere | Improvement) | Improvement) |
| H1N1 | HA | North | 31.75 | 81.32 |
| H1N1 | HA | South | 33.71 | 72.04 |
| H1N1 | HA | avg | 32.73 | 76.68 |
| H3N2 | HA | North | 39.39 | 41.38 |
| H3N2 | HA | South | 31.00 | 28.81 |
| H3N2 | HA | avg | 35.20 | 35.10 |
| H1N1 | NA | North | 22.09 | 60.00 |
| H1N1 | NA | South | 10.81 | 50.79 |
| H1N1 | NA | avg | 16.45 | 55.40 |
| H3N2 | NA | North | 28.38 | 45.95 |
| H3N2 | NA | South | 24.69 | 47.73 |
| H3N2 | NA | avg | 26.53 | 46.84 |

TABLE 2
Qnet induced distance varying for fixed sequence pair when background population changes (rows
1-5), sequences with small edit distance and large q-distance, and the converse (rows 6-9)

| | edit | | | q- | Year | Year |
|---|---|---|---|---|---|---|
| | dist. | sequence A | sequence B | distance | A* | B* |
| 1 | 18 | A/Singapore/23J/2007 | A/Tennessee/UR06-0294/2007 | 0.0111 | 2007 | 2007 |
| 2 | 18 | A/Singapore/23J/2007 | A/Tennessee/UR06-0294/2007 | 0.0094 | 2008 | 2008 |
| 3 | 18 | A/Singapore/23J/2007 | A/Tennessee/UR06-0294/2007 | 0.0027 | 2009 | 2009 |
| 4 | 18 | A/Singapore/23J/2007 | A/Tennessee/UR06-0294/2007 | 0.0025 | 2010 | 2010 |
| 5 | 18 | A/Singapore/233/2007 | A/Tennessee/UR06-0294/2007 | 0.6163 | 2007 | 2010 |
| 6 | 11 | A/Naypyitaw/M783/2008 | A/Singapore/201/2008 | 0.8852 | 2008 | 2008 |
| 7 | 15 | A/Cambodia/W0908339/2012 | A/Singapore/DMS1233/2012 | 0.2737 | 2012 | 2012 |
| 8 | 126 | A/South Dakota/03/2008 | A/Singapore/10/2008 | 0.3034 | 2008 | 2008 |
| 9 | 141 | A/Jodhpur/3248/2012 | A/Cambodia/W0908339/2012 | 0.2405 | 2012 | 2012 |

*year A and B correspond to the assumed collection years for sequences A and B respectively, for purposes of this example. Sequence A in Row 1 is collected in 2007, but is assumed to be from different years in rows 2-4 to demonstrate the change in q-distance from sequence B, arising only from a change in the background population.

TABLE 3
Correlation between q-distance and edit
distance between sequence pairs

| phenotypes | correlation |
|---|---|
| Influenza H1N1 HA | 0.76 |
| Influenza H1N1 NA | 0.74 |
| Influenza H3N2 HA | 0.85 |
| Influenza H3N2 NA | 0.79 |
| SARS-CoV-2 | 0.52 |

TABLE 4
Number of sequences collected from public databases

| | | No. of |
|---|---|---|
| Database | Strain | Sequences |
| NCBI | Influenza H1N1 HA | 7,761 |
| NCBI | Influenza H1N1 NA | 5,640 |
| NCBI | Influenza H3N2 HA | 6,568 |
| GISAID | Influenza H3N2 HA | 2,000 |
| NCBI | Influenza H3N2 NA | 4,919 |
| GISAID | Influenza H3N2 NA | 2,000 |
| NCBI | SARS-CoV-2 | 24 |
| GISAID | SARS-CoV-2 | 371 |
| NCBI | betacoronavirus (non-SARS-CoV-2) | 921 |
| Total | | 30,204 |

TABLE 5
General linear model for evaluating effect
of data diversity on Qnet performance

| variable name | description |
|---|---|
| qnet_complexity | Cumulative number of nodes in |
| | all predictors in the |
| | corresponding Qnet |
| data_diversity | Number of clusters in set of |
| | input sequence where each |
| | sequence in a specific cluster is |
| | separated by at least 5 |
| | mutations from sequences not |
| | in the cluster |
| Idistance_WHO | Deviation of WHO predicted |
| | strain from the dominant strain |

model:dev [symbol] qnet_complexity [symbol] data_diversity [symbol] qnet_complexity [symbol] data_diversity [symbol] Idistance_WHO
Generalized Linear Model Regression Results

| | | | |
|---|---|---|---|
| Dep. Variable: | [symbol] | No. Observations: | 23 [symbol] |
| Model: | [symbol] | D [symbol] Residuals: | 230 |
| Model Family: | [symbol] | D [symbol] Model: | 4 |
| Link Function: | [symbol] | Scale: | 23.214 |
| Method: | [symbol] | Log-Likelihood: | −700.43 |
| Date: | Thu, 11 [symbol] 2020 | [symbol] : | [symbol] 339.2 |
| Time: | [symbol] : [symbol] : [symbol] | [symbol] : | [symbol] |
| No. [symbol] : | 3 | Covariance Type: | nonrobust |

| | coef | std err | z | P > [symbol] | [symbol] 0.025 | 0.97 [symbol] |
|---|---|---|---|---|---|---|
| Intercept | −0.111 [symbol] | 1.0 [symbol] 0 | −0.102 | 0. [symbol] 18 | −2.24 [symbol] | 2.025 |
| qnet_complexity | 0.000 [symbol] | 0.000 | 1.075 | 0.282 | −0.000 | 0.001 |
| data_deversity | 0.31 [symbol] 7 | 0.126 | 2.531 | 0.011 | 0.072 | 0.567 |
| qnet_complexity:data_deversity | [symbol] | [symbol] | −1. [symbol] | 0.1 [symbol] 7 | −0.000 | [symbol] |
| Idistance_WHO | −0.0 [symbol] 48 | 0.03 [symbol] | −1.007 | 0.314 | −0.102 | 0.033 |

model:dev [symbol] qnet_complexity [symbol] data_diversity [symbol] Idistance_WHO
Generalized Linear Model Regression Results

| | | | |
|---|---|---|---|
| Dep. Variable: | [symbol] | No. Observations: | 23 [symbol] |
| Model: | [symbol] | D [symbol] Residuals: | 231 |
| Model Family: | [symbol] | D [symbol] Model: | 3 |
| Link Function: | [symbol] | Scale: | 23. [symbol] 06 |
| Method: | [symbol] | Log-Likelihood: | −701.41 |
| Date: | Thu, 11 [symbol] 2020 | [symbol] : | [symbol] .6 |
| Time: | [symbol] : [symbol] : [symbol] | [symbol] : | [symbol] |
| No. [symbol] : | 3 | Covariance Type: | nonrobust |

| | coef | std err | z | P > [symbol] | [symbol] 0.025 | 0.97 [symbol] |
|---|---|---|---|---|---|---|
| Intercept | 1.0841 | 0. [symbol] | 1. [symbol] 30 | 0.103 | −0.219 | 2. [symbol] 8 [symbol] |
| qnet_complexity | [symbol] | 0.000 | −0.15 [symbol] | 0. [symbol] 7 [symbol] | −0.001 | 0.000 |
| data_deversity | 0.17 [symbol] | 0.075 | 2. [symbol] 92 | 0.017 | 0.0 [symbol] 2 | 0.025 |
| Idistance_WHO | −0.0695 | 0.024 | −2. [symbol] 0 | 0.003 | −0.116 | −0.023 |

[symbol]

indicates data missing or illegible when filed

TABLE 6
H1N1 HA northern hemisphere

| | | | | WHO | Qnet |
|---|---|---|---|---|---|
| year | WHO recommendation | dominant strain | Qnet recommendation | error | error |
| 2001-2002 | A/New Caledonia/20/99 | A/Canterbury/41/2001 | A/Dunedin/2/2000 | 4 | 8 |
| 2002-2003 | A/New Caledonia/20/99 | A/Taiwan/587/2002 | A/Canterbury/41/2001 | 3 | 1 |
| 2003-2004 | A/New Caledonia/20/99 | A/Memphis/5/200 [symbol] | A/New York/291/2002 | 5 | 2 |
| 2004-2005 | A/New Caledonia/20/99 | A/Thailand/Siriraj-Rama-TT/2004 | A/Memphis/5/2003 | 7 | 4 |
| 2005-2006 | A/New Caledonia/20/99 | A/ [symbol] /217/2005 | A/Canterbury/106/2004 | 8 | 10 |
| 2006-2007 | A/New Caledonia/20/99 | A/India/ [symbol] 4980/2006 | A/ [symbol] /819/2005 | 8 | 1 |
| 2007-2008 | A/Solomon Islands/3/2006 | A/Norway/1701/2007 | A/ [symbol] /819/2005 | 8 | 11 |
| 2008-2009 | A/Brisbane/59/2007 | A/Pennsylvania/02/2008 | A/Kentucky/UR06-047 [symbol] /2007 | 2 | 2 |
| 2009-2010 | A/Brisbane/59/2007 | A/Singapore/ [symbol] /2009 | A/ [symbol] /241-2008 | 119 | 119 |
| 2010-2011 | A/California/7/2009 | A/England/01220740/2010 | A/Singapore/ON10 [symbol] 0/2009 | 5 | 1 |
| 2011-2012 | A/California/7/2009 | A/Punjab/041/2011 | A/England/01220740/2010 | 7 | 2 |
| 2012-2013 | A/California/7/2009 | A/British Columbia/ [symbol] 01/2012 | A/Punjab/041/2011 | 11 | 4 |
| 2013-2014 | A/California/7/2009 | A/Moscow/ [symbol] -32/2013 | A/ [symbol] /1 [symbol] 9/2012 | 10 | 2 |
| 2014-2015 | A/California/7/2009 | A/Thailand/C [symbol] -C51 [symbol] 9/2014 | A/Thailand/CU-C5169/2014 | 12 | [symbol] |
| 2015-2016 | A/California/7/2009 | A/Georgia/15/2015 | A/Thailand/CU-C5169/2014 | 14 | 2 |
| 2016-2017 | A/California/7/2009 | A/Hawaii/21/2016 | A/Hawaii/21/2018 | 1 [symbol] | 0 |
| 2017-2018 | A/Michigan/45/201 [symbol] | A/Michigan/291/2017 | A/Beijing- [symbol] /SWL1335/2016 | 5 | 4 |
| 2018-2019 | A/Michigan/45/201 [symbol] | A/Washington/55/2018 | A/Michigan/291/2017 | 6 | 1 |
| 2019-2020 | A/Brisbane/02/2018 | A/Kentucky/06/2019 | A/Washington/55/2018 | 5 | 1 |
| 2020-2021 | A/Hawaii/70/2019 | [symbol] 1 | A/Italy/ [symbol] 451/2018 | [symbol] 1 | [symbol] 1 |

* Dominant strain is calculated as the one closest to the centroid in the strain space that year in the edit distance metric [symbol]
indicates data missing or illegible when filed

TABLE 7
H1N1 NA southern hemisphere

| | | | | WHO | Qnet |
|---|---|---|---|---|---|
| year | WHO recommendation | dominant strain | Qnet recommendation | error | error |
| 2001-2002 | A/New Caledonia/20/99 | A/Canterbury/41/2001 | A/South Canterbury/50/2000 | 4 | 6 |
| 2002-2003 | A/New Caledonia/20/99 | A/Taiwan/ [symbol] /2002 | A/Canterbury/41/2001 | 3 | 1 |
| 2003-2004 | A/New Caledonia/20/99 | A/Memphis/5/2003 | A/New York/2 [symbol] 1/2002 | [symbol] | 2 |
| 2004-2005 | A/New Caledonia/20/99 | A/Thailand/Siriraj-Rama-TT/2004 | A/Memphis/5/2003 | 7 | 4 |
| 2005-2006 | A/New Caledonia/20/99 | A/ [symbol] /217/2005 | A/Canterbury/106/2004 | [symbol] | 10 |
| 2006-2007 | A/New Caledonia/20/99 | A/India/34980/2006 | A/ [symbol] 217/2005 | [symbol] | 2 |
| 2007-2008 | A/New Caledonia/20/99 | A/Norway/1701/2007 | A/Thailand/CU88/2008 | 14 | 3 |
| 2008-2009 | A/Solomon Islands/3/2006 | A/Pennsylvania/02/2008 | A/Kentucky/UR0 [symbol] -0476/2007 | 9 | 2 |
| 2009-2010 | A/Brisbane/59/2007 | A/Singapore/ [symbol] /2009 | A/ [symbol] /241/2008 | 119 | 119 |
| 2010-2011 | A/California/7/2009 | A/England/012207 [symbol] /2010 | A/Singapore/ON10 [symbol] 0/2009 | 5 | 1 |
| 2011-2012 | A/California/7/2009 | A/Punjab/041/2011 | A/England/01220740/2010 | 7 | 2 |
| 2012-2013 | A/California/7/2009 | A/British Columbia/ [symbol] 01/2012 | A/Punjab/041/2011 | 11 | 4 |
| 2013-2014 | A/California/7/2009 | A/Moscow/ [symbol] -32/2013 | A/India/P122045/2012 | 10 | 5 |
| 2014-2015 | A/California/7/2009 | A/Thailand/C [symbol] -C51 [symbol] 9/2014 | A/ [symbol] /SWL1382/2013 | 12 | 4 |
| 2015-2016 | A/California/7/2009 | A/Georgia/15/2015 | A/Thailand/CU-CS1 [symbol] /2014 | 14 | 2 |
| 2016-2017 | A/California/7/2009 | A/Hawaii/21/2016 | A/Georgia/15/2015 | 1 [symbol] | 2 |
| 2017-2018 | A/Michigan/45/2015 | A/Michigan/291/2017 | A/Bejing- [symbol] /SWL1 [symbol] /2016 | 5 | 4 |
| 2018-2019 | A/Michigan/45/2015 | A/Washington/ [symbol] /2018 | A/Michigan/291/2017 | 6 | 1 |
| 2019-2020 | A/Michigan/45/2015 | A/Kentucky/06/2019 | A/Washington/55/2018 | 7 | 1 |
| 2020-2021 | A/Brisbane/02/201 [symbol] | [symbol] 1 | A/Italy/8451/2019 | [symbol] 1 | [symbol] 1 |

* Dominant strain is calculated as the one closest to the centroid in the strain space that year in the edit distance metric [symbol]
indicates data missing or illegible when filed

TABLE 8
H1N1 NA northern hemisphere

| | | | | WHO | Qnet |
|---|---|---|---|---|---|
| year | WHO recommendation | dominant strain | Qnet recommendation | error | error |
| 2001-2002 | A/New Caledonia/20/99 | A/New York/447/2001 | A/Memphis/15/2000 | 4 | 4 |
| 2002-2003 | A/New Caledonia/20/99 | A/Paris/ [symbol] /2002 | A/New York/447/2001 | 1 | 5 |
| 2003-2004 | A/New Caledonia/20/99 | A/Memphis/5/2003 | A/New York/291/2002 | 3 | 5 |
| 2004-2005 | A/New Caledonia/20/99 | A/Singapore/14/2004 | A/Memphis/5/2003 | 2 | [symbol] |
| 2005-2006 | A/New Caledonia/20/99 | A/Memphis/5/200 [symbol] | A/Memphis/5/2003 | 3 | 0 |
| 2006-2007 | A/New Caledonia/20/99 | A/Massachusetts/08/2006 | A/Sofia/361/2005 | 4 | 2 |
| 2007-2008 | A/Solomon Islands/ [symbol] /2006 | A/Massachusetts/08/2006 | A/Sofia/361/2005 | [symbol] | 2 |
| 2008-2009 | A/Brisbane/59/2007 | A/Brisbane/ [symbol] /2007 | A/Maryland/04/2007 | [symbol] | [symbol] |
| 2009-2010 | A/Brisbane/59/2007 | A/Thailand/SR08021/2009 | A/Thailand/SP08207/200 [symbol] | 87 | 87 |
| 2010-2011 | A/California/7/2009 | A/Thailand/SR08021/2009 | A/ [symbol] /70 [symbol] /200 [symbol] | 2 | [symbol] |
| 2011-2012 | A/California/7/2009 | A/Tula/CRIE-GSYu/2011 | A/Thailand/SR0 [symbol] 021/200 [symbol] | 4 | 2 |
| 2012-2013 | A/California/7/2009 | A/Tula/CRIE-GSYu/2011 | A/Tula/CRIE-GSYu/2011 | 4 | 0 |
| 2013-2014 | A/California/7/2009 | A/ [symbol] /SWL1824/2013 | A/Long [symbol] /SWL [symbol] /2013 | 5 | 3 |
| 2014-2015 | A/California/7/2009 | A/Long [symbol] /SWL2 [symbol] 7/2014 | A/Utah/06/2013 | 9 | 3 |
| 2015-2016 | A/California/7/2009 | A/Michigan/45/2015 | A/ [symbol] / [symbol] 08M/2014 | 14 | 4 |
| 2016-2017 | A/California/7/2009 | A/Michigan/45/2015 | A/Michigan/45/2015 | 14 | 0 |
| 2017-2018 | A/Michigan/45/2015 | A/ [symbol] /37/2017 | A/Michigan/45/2015 | 3 | 3 |
| 2018-2019 | A/Michigan/45/2015 | A/Kenya/47/2018 | A/Kenya/47/2018 | 4 | 0 |
| 2019-2020 | A/Brisbane/02/2018 | A/Kenya/47/2018 | A/Kenya/47/2018 | 1 | 0 |
| 2020-2021 | A/Hawaii/70/2019 | [symbol] 1 | A/Kenya/47/2018 | [symbol] 1 | [symbol] 1 |

* Dominant strain is calculated as the one closest to the centroid in the strain space that year in the edit distance metric [symbol]
indicates data missing or illegible when filed

TABLE 9
H1N1 NA southern hemisphere

| | | | | WHO | Qnet |
|---|---|---|---|---|---|
| year | WHO recommendation | dominant strain | Qnet recommandations | error | error |
| 2001-2002 | A/New Caledonia/20/99 | A/New York/447/2001 | A/Canterbury/37/2000 | 4 | [symbol] |
| 2002-2003 | A/New Caledonia/20/99 | A/Paris/0 [symbol] /2002 | A/New York/447/2001 | 1 | 5 |
| 2003-2004 | A/New Caledonia/20/99 | A/Memphis/5/200 [symbol] | A/New York/291/2002 | [symbol] | [symbol] |
| 2004-2005 | A/New Caledonia/20/99 | A/Singapore/14/2004 | A/Memphis/5/2003 | 2 | 3 |
| 2005-2006 | A/New Caledonia/20/99 | A/Memphis/5/2003 | A/Canterbury/10 [symbol] /2004 | 3 | 6 |
| 2006-2007 | A/New Caledonia/20/99 | A/Massachusetts/08/2006 | A/Sofia/361/2005 | 4 | 2 |
| 2007-2008 | A/New Caledonia/20/99 | A/Massachusetts/08/2006 | A/Thailand/ [symbol] MSC-UDN-2 [symbol] /200 [symbol] | 4 | 8 |
| 2008-2009 | A/Solomon Islands/3/200 [symbol] | A/Brisbane/59/2007 | A/Tennessee/U [symbol] 06-0151/2007 | 15 | 13 |
| 2009-2010 | A/Brisbane/59/2007 | A/Thailand/SR [symbol] 8021/2009 | A/Nebraska/07/200 [symbol] | 87 | 87 |
| 2010-2011 | A/California/7/2009 | A/Thailand/SR [symbol] 8021/2009 | A/Rome/709/2009 | 2 | 9 |
| 2011-2012 | A/California/7/2009 | A/Tula/CRIE-GSYu/2011 | A/Thailand/S [symbol] 021/2009 | 4 | 2 |
| 2012-2013 | A/California/7/2009 | A/Tula/CRIE-GSYu/2011 | A/Tula/GRIE-GSYu/2011 | 4 | [symbol] |
| 2013-2014 | A/California/7/2009 | A/ [symbol] /SWL1824/2013 | A/Oman/SQUH- [symbol] /2012 | 5 | 4 |
| 2014-2015 | A/California/7/2009 | A/Long [symbol] /SWL24 [symbol] 7/2014 | A/ [symbol] /SWL1 [symbol] /2013 | [symbol] | [symbol] |
| 2015-2016 | A/California/7/2009 | A/Michigan/45/2015 | A/Long [symbol] /SWL2457/2014 | 14 | 5 |
| 2016-2017 | A/California/7/2009 | A/Michigan/45/2015 | A/Michigan/45/2015 | 14 | 0 |
| 2017-2018 | A/Michigan/45/2015 | A/ [symbol] / [symbol] /2017 | A/Michigan/45/2015 | [symbol] | [symbol] |
| 2018-2019 | A/Michigan/45/2015 | A/Kenya/47/2018 | A/Kentucky/26/2017 | 4 | 2 |
| 2019-2020 | A/Michigan/45/2015 | A/Kenya/47/2018 | A/Kenya/47/2018 | 4 | 0 |
| 2020-2021 | A/Brisbane/02/201 [symbol] | [symbol] 1 | A/Kenya/47/2018 | [symbol] 1 | [symbol] 1 |

* Dominant strain is calculated as the one closest to the centroid in the strain space that year in the edit distance metric [symbol]
indicates data missing or illegible when filed

TABLE 10
H3N2 HA northern hemisphere

| | | | | WHO | Qnet |
|---|---|---|---|---|---|
| year | WHO recommendation | dominant strain | Qnet recommendation | error | error |
| 2005-2006 | A/California/7/2004 | A/Denmark/195/200 [symbol] | A/ [symbol] / [symbol] /2004 | 10 | 2 |
| 2006-2007 | A/Wisconsin/67/2005 | A/New York/5/200 [symbol] | A/South Australia/22/2005 | 5 | 4 |
| 2007-2008 | A/Wisconsin/67/2005 | A/Tennessee/1 [symbol] /2007 | A/Colorado/05/2006 | 8 | 5 |
| 2008-2009 | A/Brisbane/10/2007 | A/Massachusetts/13/2008 | A/Tennessee/11/2007 | 3 | 2 |
| 2009-2010 | A/Brisbane/10/2007 | A/Hawaii/14/2009 | A/ [symbol] /0 [symbol] /200 [symbol] | 7 | 8 |
| 2010-2011 | A/ [symbol] /1 [symbol] /2009 | A/Utah/12/2010 | A/Hawaii/14/200 [symbol] | 8 | 7 |
| 2011-2012 | A/ [symbol] /1 [symbol] /2009 | A/ [symbol] /14202/2011 | A/Utah/12/2010 | 4 | 4 |
| 2012-2013 | A/Victoria/ [symbol] /2011 | A/ [symbol] /927/2012 | A/ [symbol] / [symbol] 5/2012 | 4 | 3 |
| 2013-2014 | A/Victoria/ [symbol] /2011 | A/Delaware/01/2013 | A/Singapore/ [symbol] 2.934/2012 | 4 | 1 |
| 2014-2015 | A/Texas/ [symbol] /2012 | A/Hong Kong/4801/2014 | A/Nebraska/0 [symbol] /2013 | 10 | 9 |
| 2015-2016 | A/Switzerland/ [symbol] /2013 | A/Hong Kong/4801/2014 | A/Hong Kong/4801/2014 | 10 | 0 |
| 2016-2017 | A/Hong Kong/4801/2014 | A/Hong Kong/4801/2014 | A/Hong Kong/4801/2014 | 0 | 0 |
| 2017-2018 | A/Hong Kong/4801/2014 | A/Maryland/25/2617 | A/New York/0 [symbol] /2016 | 3 | 1 |
| 2018-2019 | A/Singapore/INFIMH [symbol] /2016 | A/Vermont/04/2018 | A/ [symbol] /038/2017 | 8 | 5 |
| 2019-2020 | A/Kansas/14/2017 | A/Kentucky/27/2019 | A/California/7330/2018 | 1 [symbol] | 12 |
| 2020-2021 | A/Hong Kong/2671/201 [symbol] | [symbol] 1 | A/Kentucky/27/2019 | [symbol] 1 | [symbol] 1 |

* Dominant strain is calculated as the one closest to the centroid in the strain space that year in the edit distance metric [symbol]
indicates data missing or illegible when filed

TABLE 11
H3N2 HA southern hemisphere

| | | | | WHO | Qnet |
|---|---|---|---|---|---|
| year | WHO recommendation | dominant strain | Qnet recommendation | error | error |
| 2005-2006 | A/ [symbol] /1/2004 | A/Denmark/195/2005 | A/ [symbol] /21/2004 | 3 | 3 |
| 2006-2007 | A/California/7/200 [symbol] | A/New York/5/200 [symbol] | A/South Australia/22/2005 | 12 | 4 |
| 2007-2008 | A/Wisconsin/ [symbol] 7/2005 | A/Tennessee/11/2007 | A/New York/ [symbol] 23/2006 | 8 | 5 |
| 2008-2009 | A/Brisbane/10/2007 | A/Massachusetts/13/2008 | A/Tennessee/11/2007 | 3 | 2 |
| 2009-2010 | A/Brisbane/10/2007 | A/Hawaii/14/2009 | A/ [symbol] /03/2008 | 7 | 6 |
| 2010-2011 | A/ [symbol] /1 [symbol] /2009 | A/Utah/12/2010 | A/Hawaii/14/2009 | 8 | 7 |
| 2011-2012 | A/ [symbol] /1 [symbol] /2009 | A/ [symbol] /14202/2011 | A/Utah/12/2010 | 4 | 4 |
| 2012-2013 | A/ [symbol] /1 [symbol] /2009 | A/ [symbol] / [symbol] 27/2012 | A/ [symbol] /14202/2011 | [symbol] | 4 |
| 2013-2014 | A/Victoria/3 [symbol] 1/2011 | A/Delaware/01/2013 | A/ [symbol] /IPE00 [symbol] 0/2012 | 4 | 7 |
| 2014-2015 | A/Texas/50/2012 | A/Hong Kong/4801/2014 | A/Delaware/01/2013 | 10 | 7 |
| 2015-2016 | A/Switzerland/9715293/2013 | A/Hong Kong/4801/2014 | A/Hong Kong/4801/2014 | 10 | 0 |
| 2016-2017 | A/Hong Kong/4801/2014 | A/Hong Kong/4801/2014 | A/Hong Kong/4801/2014 | 0 | 0 |
| 2017-2018 | A/Hong Kong/4801/2014 | A/Maryland/25/2017 | A/Ontario/ [symbol] 2016 | 3 | 4 |
| 2018-2019 | A/Singapore/INFIMH [symbol] /2016 | A/Vermont/04/2016 | A/Ontario/ [symbol] /2017 | 8 | 5 |
| 2019-2020 | A/Switzerland/8080/2017 | A/Kentucky/27/2019 | A/California/7330/2018 | 1 [symbol] | 12 |
| 2020-2021 | A/South Australia/34/2019 | [symbol] 1 | A/Kentucky/27/2019 | [symbol] 1 | [symbol] 1 |

* Dominant strain is calculated as the one closest to the centroid in the strain space that year in the edit distance metric [symbol]
indicates data missing or illegible when filed

TABLE 12
H3N2 NA northern hemisphere

| | | | | WHO | Qnet |
|---|---|---|---|---|---|
| year | WHO recommendation | dominant strain | Qnet recommendation | error | error |
| 2003-2004 | A/Moscow/10/99 | A/Denmark/107/2003 | A/New York/101/2002 | 13 | 3 |
| 2004-2005 | A/ [symbol] /411/2002 | A/ [symbol] / [symbol] /2014 | A/New York/20/200 [symbol] | 3 | 1 [symbol] |
| 2005-2006 | A/California/7/2004 | A/Denmark/20 [symbol] /2005 | A/Denmark/203/2005 | 4 | 0 |
| 2006-2007 | A/Wisconsin/67/2005 | A/Berlin/32/2006 | A/Mexico/ [symbol] 2227/2005 | 1 | 1 |
| 2007-2008 | A/Wisconsin/67/2005 | A/Brazil/ [symbol] 0/2007 | A/ [symbol] / [symbol] 57/2005 | [symbol] | 7 |
| 2008-2009 | A/Brisbane/10/2007 | A/ [symbol] /1 [symbol] /200 [symbol] | A/Brazil/80/2007 | 3 | 2 |
| 2009-2010 | A/Brisbane/10/2007 | A/ [symbol] /1 [symbol] /200 [symbol] | A/Wisconsin/24/2008 | 3 | 1 |
| 2010-2011 | A/ [symbol] /16/200 [symbol] | A/California/17/2010 | A/New York/70/200 [symbol] | 2 | [symbol] |
| 2011-2012 | A/ [symbol] /16/200 [symbol] | A/Texas/14/2011 | A/Virginia/05/2010 | 3 | 2 |
| 2012-2013 | A/Victoria/361/2011 | A/New York/02/2012 | A/Singapore/C2011.493/2011 | 4 | 1 |
| 2013-2014 | A/Victoria/361/2011 | A/Michigan/02/2013 | A/Idaho/38/2012 | 3 | 1 |
| 2014-2015 | A/Texas/50/2012 | A/ [symbol] / [symbol] 4/2014 | A/Michigan/02/2013 | 3 | 1 |
| 2015-2016 | A/Switzerland/9715/2013 | A/ [symbol] /471/2015 | A/ [symbol] /471/2015 | [symbol] | 0 |
| 2016-2017 | A/Hong Kong/4801/2014 | A/North Carolina/62/2018 | A/ [symbol] /471/201 [symbol] | 7 | 2 |
| 2017-2018 | A/Hong Kong/4801/2014 | A/Texas/277/2017 | A/Texas/277/2017 | [symbol] | 0 |
| 2018-2019 | A/Singapore/INFIMH [symbol] /2016 | A/Japan/NHRC_FDX70 [symbol] 2/2018 | A/Netherlands/3530/2017 | 4 | 3 |
| 2019-2020 | A/Kansas/14/2017 | A/Washington/9757/2019 | | 3 | 11 |
| 2020-2021 | A/Hong Kong/2871/2019 | [symbol] 1 | A/Washington/ [symbol] 757/2019 | [symbol] 1 | [symbol] 1 |

* Dominant strain is calculated as the one closest to the centroid in the strain space that year in the edit distance metric [symbol]
indicates data missing or illegible when filed

TABLE 13
H3N2 NA southern hemisphere

| | | | | WHO | Qnet |
|---|---|---|---|---|---|
| year | WHO recommendation | dominant strain | Qnet recommendation | error | error |
| 2003-2004 | A/Moscow/10/9 [symbol] | A/Denmark/107/2003 | A/New York/101/2062 | 13 | 3 |
| 2004-2005 | A/Fujian/411/2002 | A/ [symbol] /3 [symbol] /2004 | A/New York/20/2003 | 3 | 1 [symbol] |
| 2005-2006 | A/ [symbol] /1/2004 | A/Denmark/203/2005 | A/ [symbol] /1/2004 | 2 | 2 |
| 2006-2007 | A/California/7/2004 | A/Berlin/32/200 [symbol] | A/Mexico/ [symbol] 2227/2005 | 3 | 1 |
| 2007-2008 | A/Wisconsin/07/2005 | A/Brazil/ [symbol] 0/2007 | A/Ohio/06/2006 | [symbol] | 10 |
| 2008-2009 | A/Brisbane/10/2007 | A/ [symbol] /1 [symbol] /2009 | A/Brazil/80/2007 | 3 | 2 |
| 2009-2010 | A/Brisbane/10/2007 | A/ [symbol] /1 [symbol] /2009 | A/Wisconsin/24/200 [symbol] | 3 | 1 |
| 2010-2011 | A/ [symbol] /1 [symbol] /200 [symbol] | A/California/17/2010 | A/New York/70/200 [symbol] | 2 | 3 |
| 2011-2012 | A/ [symbol] /1 [symbol] /200 [symbol] | A/Texas/14/2011 | A/Virginia/05/2010 | 3 | 2 |
| 2012-2013 | A/ [symbol] /1 [symbol] /200 [symbol] | A/New York/02/2012 | A/Texas/14/2011 | 4 | 1 |
| 2013-2014 | A/Victoria/ [symbol] /2011 | A/Michigan/02/2013 | A/New York/02/2012 | [symbol] | 3 |
| 2014-2015 | A/Texas/50/2012 | A/ [symbol] / [symbol] 9634/2014 | A/Michigan/02/2013 | [symbol] | 1 |
| 2015-2016 | A/Switzerland/97152 [symbol] /2013 | A/ [symbol] /471/2015 | A/ [symbol] / [symbol] 4/2014 | [symbol] | 2 |
| 2016-2017 | A/Hong Kong/4801/2014 | A/North Carolina/62/2016 | A/ [symbol] /471/2015 | 7 | 2 |
| 2017-2018 | A/Hong Kong/4801/2014 | A/Texas/277/2017 | A/Texas/277/2017 | 8 | 0 |
| 2018-2019 | A/Singapore/INFIMH [symbol] /201 [symbol] | A/Japan/NHBC_FDX70352/201 [symbol] | A/Texas/277/2017 | 4 | 3 |
| 2019-2020 | A/Switzerland/8060/2017 | A/Washington/ [symbol] 757/2019 | A/Pennsylvania/317/2018 | 10 | 10 |
| 2020-2021 | A/South Australia/34/2019 | [symbol] 1 | A/Washington/ [symbol] 757/2019 | [symbol] 1 | [symbol] 1 |

* Dominant strain is calculated as the one closest to the centroid in the strain space that year in the edit distance metric [symbol]
indicates data missing or illegible when filed

TABLE 14
H1N1 NA southern hemisphere (multi-cluster)

| | | Qnet | Qnet | WHO | | |
|---|---|---|---|---|---|---|
| year | WHO recommendation | error0 | error1 | error | Qnet recommendation_0 | Qnet recommendation_1 |
| 2001-2002 | A/New Caledonia/20/99 | 1 | [symbol] | 4 | A/New South [symbol] /2 [symbol] /2000 | A/Canterbury/37/2000 |
| 2002-2003 | A/New Caledonia/20/99 | 0 | [symbol] | 1 | A/ [symbol] /0 [symbol] /2002 | A/New York/447/2001 |
| 2003-2004 | A/New Caledonia/20/99 | 2 | [symbol] | [symbol] | A/ [symbol] /0 [symbol] /2002 | A/ [symbol] /141/2002 |
| 2004-2005 | A/New Caledonia/20/99 | [symbol] | 4 | 2 | A/Memphis/5/200 [symbol] | A/ [symbol] /1004/2003 |
| 2005-2006 | A/New Caledonia/20/99 | 0 | 1 | [symbol] | A/Memphis/5/200 [symbol] | A/Massachusetts/0 [symbol] /200 [symbol] |
| 2006-2007 | A/New Caledonia/20/99 | 2 | [symbol] | 4 | A/ [symbol] /361/200 [symbol] | A/ [symbol] /11/200 [symbol] |
| 2007-2008 | A/New Caledonia/20/99 | 4 | 8 | 4 | A/New [symbol] /20/ [symbol] | A/New York/ [symbol] /2006 |
| 2008-2009 | A/Solomon Islands/3/2006 | 13 | 19 | 15 | A/Tennessee/UR0 [symbol] 0151/2007 | A/ [symbol] / [symbol] 0178/2007 |
| 2009-2010 | A/Brisbane/59/2007 | 88 | 90 | 87 | A/ [symbol] /TU [symbol] /2008 | A/Japan/ [symbol] /2008 |
| 2010-2011 | A/California/7/2009 | 1 | 6 | 2 | A/South Carolina/WRAIR1 [symbol] /2009 | A/Wisconsin/ [symbol] /2009 |
| 2011-2012 | A/California/7/2009 | 1 | 3 | 4 | A/England/21 [symbol] 33/2010 | A/ [symbol] /178/2010 |
| 2012-2013 | A/California/7/2009 | 1 | 22 | 4 | A/ [symbol] / [symbol] BLP/2011 | A/Rio [symbol] /57 [symbol] /2011 |
| 2013-2014 | A/California/7/2009 | 4 | 13 | [symbol] | A/Thailand/MR10 [symbol] 0/2012 | A/ [symbol] / [symbol] /2012 |
| 2014-2015 | A/California/7/2009 | 3 | 7 | 9 | A/Minnesota/02/201 [symbol] | A/ [symbol] /430/201 [symbol] |
| 2015-2016 | A/California/7/2009 | 4 | 7 | 14 | A/ [symbol] / [symbol] M/2014 | A/Virginia/NHRC- [symbol] /2014 |
| 2016-2017 | A/California/7/2009 | 0 | 3 | 14 | A/Michigan/ [symbol] /2015 | A/Colorado/30/201 [symbol] |
| 2017-2018 | A/Michigan/ [symbol] /2015 | 3 | 8 | [symbol] | A/Michigan/ [symbol] /2015 | A/Arizona/ [symbol] /201 [symbol] |
| 2018-2019 | A/Michigan/ [symbol] /2015 | 0 | 4 | 4 | A/Kenya/47/2018 | A/Michigan/ [symbol] /201 [symbol] |
| 2019-2020 | A/Michigan/ [symbol] /2015 | 0 | 2 | 4 | A/Kenya/47/2018 | A/Colorado/ [symbol] /2018 |
| 2020-2021 | A/Brisbane/02/201 [symbol] | [symbol] 1 | [symbol] 1 | 1 [symbol] | A/California/ [symbol] _BOXA- [symbol] /2019 | A/Indiana/ [symbol] /2019 |

* Dominant strain is calculated as the one closest to the centroid in the strain space that year in the edit distance metric [symbol]
indicates data missing or illegible when filed

TABLE 15
H3N2 NA southern hemisphere (multi-cluster)

| | | Qnet | Qnet | WHO | | |
|---|---|---|---|---|---|---|
| year | WHO recommendation | error0 | error1 | error | Qnet recommendation_0 | Qnet recommendation_1 |
| 2003-2004 | A/Moscow/10/ [symbol] | 4 | 5 | 1 [symbol] | A/Auckland/612/2002 | A/New York/87/2002 |
| 2004-2005 | A/Fujian/411/2002 | 1 [symbol] | 18 | [symbol] | A/New York/20/200 [symbol] | A/New York/12/2003 |
| 2005-2006 | A/ [symbol] /1/2004 | 1 | 7 | 2 | A/New York/358/2004 | A/Singapore/36/2004 |
| 2006-2007 | A/California/7/2004 | [symbol] | [symbol] | [symbol] | A/ [symbol] / [symbol] 57/2005 | A/Hong Kong/ [symbol] /200 [symbol] |
| 2007-2008 | A/Wisconsin/07/2005 | 0 | 10 | 8 | A/Brazil/ [symbol] 0/2007 | A/Wisconsin/44/2006 |
| 2008-2009 | A/Brisbane/10/2007 | 4 | 1 [symbol] | [symbol] | A/Missouri/0 [symbol] /2007 | A/Japan/72/2007 |
| 2009-2010 | A/Brisbane/10/2007 | 1 | 7 | 3 | A/Wisconsin/24/2008 | A/Mississippi/ [symbol] -0042/2008 |
| 2010-2011 | A/ [symbol] /1 [symbol] /200 [symbol] | 3 | 8 | 2 | A/New York/ [symbol] 0/2009 | A/Japan/88 [symbol] /2009 |
| 2011-2012 | A/ [symbol] /1 [symbol] /200 [symbol] | 2 | 2 | 3 | A/California/19/2010 | A/Virginia/05/2010 |
| 2012-2013 | A/ [symbol] /1 [symbol] /200 [symbol] | 1 | 12 | 4 | A/Texas/14/2011 | A/Singapore/ [symbol] /2011 |
| 2013-2014 | A/Victoria/ [symbol] /2011 | 1 | 5 | 3 | A/Idaho/ [symbol] 8/2012 | A/ [symbol] / [symbol] /2012 |
| 2014-2015 | A/Texas/50/2012 | 1 | 1 | 3 | A/Nevada/0 [symbol] /2013 | A/Michigan/02/201 [symbol] |
| 2015-2016 | A/Switzerland/97152 [symbol] /2013 | 0 | 4 | 3 | A/ [symbol] /471/2015 | A/Iran/ [symbol] 124 [symbol] /2014 |
| 2016-2017 | A/Hong Kong/ [symbol] 01/2014 | 1 | 25 | 7 | A/New Jersey/13/2015 | A/California/NHRC_BRD [symbol] 105 [symbol] /2015 |
| 2017-2018 | A/Hong Kong/ [symbol] 01/2014 | 1 | 4 | [symbol] | A/Texas/277/2017 | A/Victoria/868/2016 |
| 2018-2019 | A/Singapore/INFIMH [symbol] /201 [symbol] | 2 | 4 | [symbol] | A/ [symbol] /3580/2017 | A/Washington/17/2017 |
| 2019-2020 | A/Switzerland/8086/2017 | 4 | 10 | 1 [symbol] | A/England/ [symbol] 38/201 [symbol] | A/California/BRD12490N/2018 |
| 2020-2021 | A/South Australia/34/2019 | [symbol] 1 | [symbol] 1 | [symbol] 1 | A/South Australia/34/2019 | A/Washington/9757/2019 |

* Dominant strain is calculated as the one closest to the centroid in the strain space that year in the edit distance metric [symbol]
indicates data missing or illegible when filed

TABLE 16
Neighbors at the edge of emergence

| accession | country | date | qdistance* | host | log-likelihood bound^{†} |
|---|---|---|---|---|---|
| MG197717 | China | 2015 Jul. 6 | 0.5994 | human(Human coronavirus OC43) | −344680279.6919 |
| MG 197719 | China | 2015 Jun. 4 | 0.5595 | human(Human coronavirus OC43) | −344700037.5225 |
| MG 197710 | China | 2015 May 6 | 0.6002 | human(Human coronavirus OC43) | −345145082.3718 |
| MH940245 | Thailand | 2017 Jun. 4 | 0.6017 | human(Human coronavirus HKU1) | −346005398.0759 |
| MG197711 | China | 2015 Jun. 9 | 0.6035 | human(Human coronavirus OC43) | −347017496.9155 |
| MG197716 | China | 2015 Jun. 6 | 0.6053 | human(Human coronavirus OC43) | −348034981.4953 |
| MG197715 | China | 2015 May 21 | 0.6053 | human(Human coronavirus OC43) | −348055196.8970 |
| KF294457 | China | 2012 Jan. 1 | 0.6058 | Rhinolophus monoceros | −348315726.8189 |
| KJ473822 | China | 2012 Jan. 1 | 0.6059 | Tylonycteris pachypus | −348379385.2394 |
| MK211376 | China | 2016 Sep. 1 | 0.6065 | Rhinolophus affinis | −348745110.7506 |
| MH002342 | China | 2013 Jun. 3 | 0.6065 | Pipistrellus bat coronavirus HKU5 | −348745431.2254 |
| KJ473816 | China | 2013 Jan. 1 | 0.6066 | Rhinolophus sinicus | −348779627.4654 |
| KJ473812 | China | 2013 Jan. 1 | 0.6066 | Rhinolophus ferrumequinum | −348807413.3783 |
| MG772933 | China | 2017 Feb. 1 | 0.6066 | Rhinolophus sinicus | −348814549.9518 |
| MK211379 | China | 2016 Sep. 1 | 0.6067 | Rhinolophus affinis | −348846490.8570 |
| MK211375 | China | 2016 Sep. 1 | 0.6067 | Rhinolophus affinis | −348867989.3104 |
| MK211374 | China | 2016 Aug. 1 | 0.6068 | Rhinolophus sp. | −348893681.6418 |
| KJ473821 | China | 2014 May 6 | 0.6071 | Vespertilio superans | −349070089.5700 |
| KF569996 | China | 2011 Jan. 1 | 0.6095 | Rhinolophus affinis | −340440764.5785 |
| MN611520 | China | 2018 Mar. 1 | 0.6095 | Pipistrellus abramus | −350452309.6142 |
| KP886809 | China | 2013 May 23 | 0.6095 | Rhinolophus Ferrumequinum | −350486988.0164 |
| KP886808 | China | 2013 May 23 | 0.6095 | Rhinolophus Ferrumequinum | −350486988.0164 |
| MN611519 | China | 2018 Mar. 1 | 0.6097 | Tylonycteris pachypus | −350572065.7797 |
| NC_025217 | China | 2013 Apr. 20 | 0.6097 | Hipposideros pratti | −350580907.8832 |
| MK211377 | China | 2016 Sep. 1 | 0.6106 | Rhinolophus affinis | −351127765.1568 |
| KJ473820 | China | 2013 Jan. 1 | 0.6118 | Pipistrellus abramus | −351798100.8827 |
| MN996532^{‡} | China | 2013 Jul. 24 | 0.6155 | Rhinolophus affinis | −353944009.5536 |
| MH002341 | China | 2014 Jun. 28 | 0.6167 | Pipistrellus bat coronavirus HKU5 | −354632651.3696 |
| MH687968 | Viet Nam | 2014 Nov. 14 | 0.6174 | Rattus argentiventer | −355004271.8441 |
| MH687978 | Viet Nam | 2015 Feb. 4 | 0.6183 | Rattus argentiventer | −355553631.3715 |
| MH687969 | Viet Nam | 2014 Nov. 12 | 0.6184 | Rattus argentiventer | −355566733.0149 |
| KF294372 | China | 2011 Jan. 1 | 0.6185 | Niviventer confucianus | −355664120.7144 |
| MH687974 | Viet Nam | 2014 Nov. 12 | 0.6187 | Rattus argentiventer | −355732457.2680 |
| MH687973 | Viet Nam | 2014 Nov. 12 | 0.6189 | Rattus argentiventer | −355892765.0568 |
| MH687972 | Viet Nam | 2014 Nov. 12 | 0.6190 | Rattus argentiventer | −355956649.5930 |
| KF294370 | China | 2013 Jan. 1 | 0.6192 | Rattus tanezumi | −356024166.0313 |
| KF294371 | China | 2013 Jan. 1 | 0.6192 | Rattus losea | −356040368.5036 |
| MH687971 | Viet Nam | 2014 Nov. 12 | 0.6194 | Rattus argentiventer | −356161570.0562 |
| MH687977 | Viet Nam | 2015 Feb. 4 | 0.6199 | Rattus argentiventer | −356466591.1490 |
| KF294357 | China | 2011 Jan. 1 | 0.6214 | Apodemus agrarius | −357298941.1683 |
| KM349744 | China | 2012 May 17 | 0.6219 | Rattus norvegicus (Norway rat) | −357570433.8433 |
| NC_026011 | China | 2012 May 17 | 0.6219 | Rattus norvegicus (Norway rat) | −357570433.8433 |
| KM349743 | China | 2012 May 17 | 0.6220 | Rattus norvegicus (Norway rat) | −357646895.7536 |

*qdistance: Smaller values imply higher risk
^{†}Likelihood lower bound: Larger values implies higher risk
^{‡}RaTG13

TABLE 17
Numbering conversion to PDM09 and H3 schemes

| Query | [symbol] | [symbol] |
|---|---|---|
| 1 | — | — |
| 2 | — | — |
| 3 | — | — |
| 4 | — | — |
| 5 | — | — |
| 6 | — | — |
| 7 | — | — |
| 8 | — | — |
| 9 | — | — |
| 10 | — | — |
| 11 | — | — |
| 12 | — | — |
| 13 | — | — |
| 14 | — | — |
| 15 | — | — |
| 16 | — | — |
| 17 | — | — |
| — | — | 1 |
| — | — | 2 |
| — | — | 3 |
| — | — | 4 |
| — | — | 5 |
| — | — | 6 |
| — | — | 7 |
| — | — | 8 |
| — | — | 9 |
| — | — | 10 |
| 18 | 1 | 11 |
| 19 | 2 | 12 |
| 20 | 3 | 13 |
| 21 | 4 | 14 |
| 22 | 5 | 15 |
| 23 | 6 | 16 |
| 24 | 7 | 17 |
| 25 | 8 | 18 |
| 26 | 9 | 19 |
| 27 | 10 | 20 |
| 28 | 11 | 21 |
| 29 | 12 | 22 |
| 30 | 13 | 23 |
| 31 | 14 | 24 |
| 32 | 15 | 25 |
| 33 | 16 | 26 |
| 34 | 17 | 27 |
| 35 | 18 | 28 |
| 36 | 19 | 29 |
| 37 | 20 | 30 |
| 38 | 21 | 31 |
| 39 | 22 | 32 |
| 40 | 23 | 33 |
| 41 | 24 | 34 |
| 42 | 25 | 35 |
| 43 | 26 | 36 |
| 44 | 27 | 37 |
| 45 | 28 | 38 |
| 46 | 29 | 39 |
| 47 | 30 | 40 |
| 48 | 31 | 41 |
| 49 | 32 | 42 |
| 50 | 33 | 43 |
| 51 | 34 | 44 |
| 52 | 35 | 45 |
| 53 | 36 | 46 |
| 54 | 37 | 47 |
| 55 | 38 | 48 |
| 56 | 39 | 49 |
| 57 | 40 | 50 |
| 58 | 41 | 51 |
| 59 | 42 | 52 |
| 60 | 43 | 53 |
| 61 | 44 | 54 |
| 62 | 45 | — |
| 63 | 46 | 55 |
| 64 | 47 | 56 |
| 65 | 48 | 57 |
| 66 | 49 | 58 |
| 67 | 50 | 59 |
| 68 | 51 | 60 |
| — | — | — |
| — | — | — |
| — | — | — |
| — | — | — |
| — | — | — |
| 69 | 52 | 61 |
| 70 | 53 | 62 |
| 71 | 54 | 63 |
| 72 | 55 | 64 |
| 73 | 56 | 65 |
| 74 | 57 | 66 |
| 75 | 58 | 67 |
| 77 | 60 | 69 |
| 78 | 61 | 70 |
| 79 | 62 | 71 |
| 80 | 63 | 72 |
| 81 | 64 | 73 |
| 82 | 65 | 74 |
| 83 | 66 | 75 |
| 84 | 67 | 76 |
| 85 | 68 | 77 |
| 86 | 69 | 78 |
| 87 | 70 | 79 |
| 88 | 71 | 80 |
| 89 | 72 | 81 |
| 90 | 73 | 82 |
| 91 | 74 | — |
| 92 | 75 | 83 |
| 93 | 76 | 84 |
| 94 | 77 | 85 |
| 95 | 78 | 86 |
| 96 | 79 | 87 |
| 97 | 80 | 88 |
| 98 | 81 | 89 |
| 99 | 82 | 90 |
| 100 | 83 | 91 |
| 101 | 84 | 92 |
| 102 | 85 | — |
| 103 | 86 | 93 |
| 104 | 87 | 94 |
| 105 | 88 | 95 |
| 106 | 89 | 96 |
| 107 | 90 | 97 |
| 108 | 91 | 98 |
| 109 | 92 | 99 |
| 110 | 93 | 100 |
| 111 | 94 | 101 |
| 112 | 95 | 102 |
| — | — | — |
| — | — | — |
| 113 | 96 | 103 |
| 114 | 97 | 104 |
| 115 | 98 | 105 |
| 116 | 99 | 106 |
| 117 | 100 | 107 |
| 118 | 101 | 108 |
| 119 | 102 | 109 |
| 120 | 103 | 110 |
| 121 | 104 | 111 |
| 122 | 105 | 112 |
| 123 | 106 | 113 |
| 124 | 107 | 114 |
| 125 | 108 | 115 |
| 126 | 109 | 116 |
| 127 | 110 | 117 |
| 128 | 111 | 118 |
| 129 | 112 | 119 |
| 130 | 113 | 120 |
| 131 | 114 | 121 |
| 132 | 115 | 122 |
| 133 | 116 | 123 |
| — | — | — |
| — | — | — |
| 134 | 117 | 124 |
| 135 | 118 | 125 |
| 136 | 119 | — |
| 137 | 120 | — |
| 138 | 121 | — |
| 139 | 122 | 126 |
| 140 | 123 | 127 |
| 141 | 124 | 128 |
| — | — | — |
| — | — | — |
| — | — | — |
| — | — | — |
| — | — | — |
| 142 | 125 | 129 |
| 143 | 126 | 130 |
| 144 | 127 | 131 |
| 145 | 128 | 132 |
| 146 | 129 | 133 |
| 147 | 130 | — |
| 148 | 131 | 134 |
| 149 | 132 | 135 |
| 150 | 133 | 136 |
| 151 | 134 | 137 |
| 152 | 135 | 138 |
| 153 | 136 | 139 |
| 154 | 137 | 140 |
| 155 | 138 | 141 |
| — | — | — |
| 156 | 139 | 142 |
| 157 | 140 | 143 |
| 158 | 141 | 144 |
| 159 | 142 | 145 |
| 160 | 143 | 146 |
| 161 | 144 | 147 |
| 162 | 145 | 148 |
| 163 | 146 | 149 |
| 164 | 147 | 150 |
| 165 | 148 | 151 |
| 166 | 149 | 152 |
| 167 | 150 | 153 |
| 168 | 151 | 154 |
| 169 | 152 | 155 |
| 170 | 153 | 156 |
| 171 | 154 | 157 |
| 172 | 155 | 158 |
| — | — | — |
| — | — | — |
| — | — | — |
| — | — | — |
| 173 | 156 | 159 |
| 174 | 157 | 160 |
| 175 | 158 | 161 |
| 176 | 159 | 162 |
| 177 | 160 | 163 |
| 178 | 161 | 164 |
| 179 | 162 | 165 |
| 180 | 163 | 166 |
| 181 | 164 | 167 |
| 182 | 165 | 168 |
| 183 | 166 | 169 |
| 184 | 167 | 170 |
| — | — | — |
| 185 | 168 | 171 |
| 186 | 169 | 172 |
| 187 | 170 | 173 |
| — | — | — |
| 188 | 171 | 174 |
| 189 | 172 | 175 |
| 190 | 173 | 176 |
| 191 | 174 | 177 |
| 192 | 175 | 178 |
| 193 | 176 | 179 |
| 194 | 177 | 180 |
| 195 | 178 | 181 |
| 196 | 179 | 182 |
| 197 | 180 | 183 |
| 198 | 181 | 184 |
| 199 | 182 | 185 |
| 200 | 183 | 186 |
| 201 | 184 | 187 |
| 202 | 185 | 188 |
| 203 | 186 | 189 |
| 204 | 187 | 190 |
| 205 | 188 | 191 |
| 206 | 189 | 192 |
| 207 | 190 | 193 |
| 208 | 191 | 194 |
| 209 | 192 | 195 |
| 210 | 193 | 196 |
| 211 | 194 | 197 |
| 212 | 195 | 198 |
| 213 | 196 | 199 |
| — | — | — |
| 214 | 197 | 200 |
| 215 | 198 | 201 |
| 216 | 199 | 202 |
| 217 | 200 | 203 |
| 218 | 201 | 204 |
| 219 | 202 | 205 |
| 220 | 203 | 206 |
| 221 | 204 | 207 |
| 222 | 205 | 208 |
| 223 | 206 | 209 |
| 224 | 207 | 210 |
| 225 | 208 | 211 |
| 226 | 209 | 212 |
| 227 | 210 | 213 |
| 228 | 211 | 214 |
| 229 | 212 | 215 |
| 230 | 213 | 216 |
| 231 | 214 | 217 |
| 232 | 215 | 218 |
| 233 | 216 | 219 |
| 234 | 217 | 220 |
| 235 | 218 | 221 |
| 236 | 219 | 222 |
| 237 | 220 | 223 |
| — | — | — |
| — | — | — |
| — | — | — |
| — | — | — |
| — | — | — |
| 238 | 221 | 224 |
| 239 | 222 | 225 |
| 240 | 223 | 226 |
| 241 | 224 | 227 |
| 242 | 225 | 228 |
| 243 | 226 | 229 |
| 244 | 227 | 230 |
| 245 | 228 | 231 |
| 246 | 229 | 232 |
| 247 | 230 | 233 |
| 248 | 231 | 234 |
| 249 | 232 | 235 |
| 250 | 233 | 236 |
| 251 | 234 | 237 |
| 252 | 235 | 238 |
| 253 | 236 | 239 |
| 254 | 237 | 240 |
| 255 | 238 | 241 |
| 256 | 239 | 242 |
| 257 | 240 | 243 |
| 258 | 241 | 244 |
| 259 | 242 | 245 |
| 260 | 243 | 246 |
| 261 | 244 | 247 |
| 262 | 245 | 248 |
| 263 | 246 | 249 |
| 264 | 247 | 250 |
| 265 | 248 | 251 |
| 266 | 249 | 252 |
| 267 | 250 | 253 |
| 268 | 251 | 254 |
| 269 | 252 | 255 |
| 270 | 253 | 256 |
| 271 | 254 | 257 |
| 272 | 255 | 258 |
| 273 | 256 | 259 |
| 274 | 257 | 260 |
| 275 | 258 | 261 |
| 276 | 259 | 262 |
| — | — | — |
| — | — | — |
| — | — | — |
| — | — | — |
| — | — | — |
| — | — | — |
| — | — | — |
| — | — | — |
| — | — | — |
| 277 | 260 | — |
| 278 | 261 | 263 |
| 279 | 262 | 264 |
| 280 | 263 | 265 |
| 281 | 264 | 266 |
| 282 | 265 | 267 |
| 283 | 266 | 268 |
| 284 | 267 | 269 |
| 285 | 268 | 270 |
| 286 | 269 | 271 |
| 287 | 270 | 272 |
| 288 | 271 | 273 |
| 289 | 272 | 274 |
| 290 | 273 | 275 |
| 291 | 274 | 276 |
| 292 | 275 | 277 |
| 293 | 276 | 278 |
| 294 | 277 | 279 |
| 295 | 278 | 280 |
| 296 | 279 | 281 |
| 297 | 280 | 282 |
| 298 | 281 | 283 |
| 299 | 282 | 284 |
| 300 | 283 | 285 |
| — | — | — |
| 301 | 284 | 286 |
| 302 | 285 | 287 |
| 303 | 286 | 288 |
| 304 | 287 | 289 |
| 305 | 288 | 290 |
| 306 | 289 | 291 |
| 307 | 290 | 292 |
| 308 | 291 | 293 |
| 309 | 292 | 294 |
| 310 | 293 | 295 |
| 311 | 294 | 296 |
| — | — | — |
| 312 | 295 | 297 |
| 313 | 296 | 298 |

[symbol]

indicates data missing or illegible when filed

What is claimed is:

1. A method comprising:
receiving a first plurality of aligned genomic sequences of a virus from a database, the aligned genomic sequences having a first common background; and
calculating a Qnet for each genomic sequence of the first plurality of aligned genomic sequences by:
calculating a conditional inference tree for each index of the aligned genomic sequences using other indices in the aligned genomic sequences as predictive features; and
calculating predictors for indices that were used as predictive features when calculating the conditional inference tree for each index.

2. The method of claim 1, wherein the first common background of the first plurality of aligned genomic sequences comprises a common year of collection.

3. The method of claim 1, wherein the first common background of the first plurality of aligned genomic sequences comprises a common species from which the aligned genomic sequences were collected.

4. The method of claim 1, further comprising calculating distances between pairs of sequences of the first plurality of aligned genomic sequences based on the Qnet.

5. The method of claim 4, wherein calculating the distances comprises calculating q-distances as the square root of the Jensen-Shannon divergence of conditional nucleotide distributions from the Qnet for a sequence to conditional nucleotide distributions from the Qnet for a different sequence.

6. The method of claim 5, further comprising predicting a future dominant strain of the virus based on the calculated q-distances.

7. The method of claim 6, wherein predicting the future dominant strain of the virus comprises determining which sequence of the plurality of aligned genomic sequences has a smallest q-distance from a current dominant strain that is a member of the plurality of aligned genomic sequences.

8. The method of claim 5, further comprising calculating Qnets for a second plurality of aligned genomic sequences of the virus, the second plurality of aligned genomic sequences having a second common background different than the first common background of the first plurality of aligned genomic sequences.

9. The method of claim 8, further comprising calculating q-distances from genomic sequences of the first plurality of aligned genomic sequences to genomic sequences of the second plurality of aligned genomic sequences.

10. The method of claim 9, wherein the first common background comprises a first species, the second common background comprises a second species, and further comprising calculating a probability of the virus jumping from the first species to the second species based on the calculated q-distances from genomic sequences of the first plurality of aligned genomic sequences to genomic sequences of the second plurality of aligned genomic sequences.

11. A system comprising:
a processor; and
a memory, the memory storing instructions that, when executed by the processor, cause the processor to:
receive a first plurality of aligned genomic sequences of a virus from a database, the aligned genomic sequences having a first common background; and
calculate a Qnet for each genomic sequence of the first plurality of aligned genomic sequences by:
calculating a conditional inference tree for each index of the aligned genomic sequences using other indices in the aligned genomic sequences as predictive features; and
calculating predictors for indices that were used as predictive features when calculating the conditional inference tree for each index.


SEQUENCE LISTING (20 sequences)

SEQ ID NO: 1 - DNA, 50 bp, Influenza A virus
ggaaaacaaaagcaacaaaatgaaaaaagacgccagttcattggtactgg

SEQ ID NO: 2 - DNA, 50 bp, Influenza A virus
agcaaaagcaggggaaaacagttcaaccacctattcaactgccgccagtt

SEQ ID NO: 3 - DNA, 50 bp, Influenza A virus
agcaaaagcaggggaaaacagtttaaccacctattcaactgtcgccagtt

SEQ ID NO: 4 - DNA, 50 bp, Influenza A virus
atgaagactatcattgctttaccttgagaagtgttgctttgttggggttc

SEQ ID NO: 5 - protein, 250 aa, Influenza A virus
GVAPLHLGKCNIAGWILGNPECESLSTASSWSYIVETSNSDNGTCYPGDFINYEELREQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNI

SEQ ID NO: 6 - protein, 250 aa, Influenza A virus
GVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNV

SEQ ID NO: 7 - protein, 250 aa, Influenza A virus
GVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNV

SEQ ID NO: 8 - protein, 5 aa, Influenza A virus
  misc_feature (3)..(3): Xaa can be any naturally occurring amino acid
GTXAV

SEQ ID NO: 9 - protein, 5 aa, Influenza A virus
RASPI

SEQ ID NO: 10 - protein, 5 aa, Influenza A virus
RTSPI

SEQ ID NO: 11 - protein, 4 aa, Influenza A virus
SSSI

SEQ ID NO: 12 - protein, 4 aa, Influenza A virus
RTPV

SEQ ID NO: 13 - protein, 4 aa, Influenza A virus
RTSV

SEQ ID NO: 14 - protein, 16 aa, Influenza A virus
APSDSKPSSIAKIESE

SEQ ID NO: 15 - protein, 16 aa, Influenza A virus
TSNNNQSTTTTEVKNK

SEQ ID NO: 16 - protein, 14 aa, Influenza A virus
LPDFKPSSAKIESE

SEQ ID NO: 17 - protein, 14 aa, Influenza A virus
LSNFQSTTTEVKNK

SEQ ID NO: 18 - protein, 14 aa, Influenza A virus
PSNVKSTTAEVKNK

SEQ ID NO: 19 - protein, 10 aa, Influenza A virus
LASGNSKQRD

SEQ ID NO: 20 - protein, 10 aa, Influenza A virus
ITARSYTHKN